(19) United States
(12) Patent Application Publication
(10) Pub. No.: US 2020/0075143 A1
(43) Pub. Date: Mar. 5, 2020

(54) MEDICAL INFORMATION MANAGEMENT SYSTEM, MEDICAL INFORMATION MANAGEMENT APPARATUS, AND MEDICAL INFORMATION MANAGEMENT METHOD

(71) Applicant: Canon Medical Systems Corporation, Otawara-shi (JP)

(72) Inventors: Sayaka TAKIKAWA, Nasushiobara (JP); Kei Yamaji, Nasushiobara (JP); Takayuki Kojima, Utsunomiya (JP); Yo Sasaki, Nasushiobara (JP); Sinya Sato, Nasushiobara (JP); Daisuke Suzuki, Otawara (JP); Mitsuo Sekine, Nasushiobara (JP)

(73) Assignee: Canon Medical Systems Corporation, Otawara-shi (JP)

(21) Appl. No.: 16/560,635

(22) Filed: Sep. 4, 2019

(30) Foreign Application Priority Data
Sep. 4, 2018 (JP) 2018-165438
Sep. 4, 2019 (JP) 2019-160932

(51) Int. Cl. G16H 10/60; G16H 40/20; G16H 30/20

(52) U.S. Cl. CPC G16H 10/60; G16H 30/20; G16H 40/20

(57) ABSTRACT

A medical information management system according to an embodiment includes a storage device and a medical information management apparatus. The storage device stores therein pieces of medical information of patients acquired by an extra-order device that acquires medical information without following medical orders issued by a hospital. The medical information management apparatus is configured to obtain medical information of a patient who is to use the hospital, from among the pieces of medical information stored in the storage device. The medical information management apparatus includes a processing circuit configured: to obtain the medical information of the patient who is to use the hospital, from among the pieces of medical information stored in the storage device; to obtain patient identification information uniquely identifying, within the hospital, the patient who is to use the hospital, and to further bring the obtained medical information of the patient into association with the patient identification information.

### CROSS-REFERENCE TO RELATED APPLICATIONS

This application is based upon and claims the benefit of priority from Japanese Patent Application No. 2018-165438, filed on Sep. 4, 2018 and Japanese Patent Application No. 2019-160932, filed on Sep. 4, 2019; the entire contents of which are incorporated herein by reference.

### FIELD

Embodiments described herein relate generally to a medical information management system, a medical information management apparatus, and a medical information management method.

### BACKGROUND

There has been a demand that, when a patient is transported to a hospital by an ambulance or the like, medical information (e.g., images and/or vital data of the patient) obtained in the vehicle during the transport be usable at the hospital being the transport destination. To meet this demand, a technique is known by which, for example, the medical information obtained in the vehicle during the transport is brought into association with medical examination data obtained at the hospital being the transport destination, on the basis of identification information of the patient assigned before the transport to the hospital.

### BRIEF DESCRIPTION OF THE DRAWINGS

FIG. 1 is a diagram illustrating an exemplary configuration of a medical information management system;

FIG. 2 is a drawing for explaining a process performed before arrival of a patient according to a first embodiment;

FIG. 3 is a drawing for explaining a process performed after the arrival of the patient according to the first embodiment;

FIG. 4 is a block diagram of a medical information management system according to the first embodiment;

FIG. 5 is a drawing illustrating an example of a patient table according to the first embodiment;

FIG. 6 is a drawing illustrating an example of an image table according to the first embodiment;

FIG. 7 is a drawing illustrating another example of the image table according to the first embodiment;

FIG. 8 is a block diagram illustrating a part of a hospital information system according to the first embodiment;

FIG. 9 is a drawing illustrating an example of a Digital Imaging and Communications in Medicine (DICOM) table according to the first embodiment;

FIG. 10 is a sequence chart illustrating an example of the process performed before the arrival of the patient according to the first embodiment;

FIG. 11 is a sequence chart illustrating an example of the process performed after the arrival of the patient according to the first embodiment;

FIG. 12 is a drawing for explaining a process performed before arrival of a patient according to a second embodiment;

FIG. 13 is a drawing for explaining a process performed after the arrival of the patient according to the second embodiment;

FIG. 14 is a drawing illustrating an example of a patient table according to the second embodiment;

FIG. 15 is a drawing illustrating an example of an image table according to the second embodiment;

FIG. 16 is a sequence chart illustrating an example of the process performed before the arrival of the patient according to the second embodiment;

FIG. 17 is a sequence chart illustrating an example of the process performed after the arrival of the patient according to the second embodiment;

FIG. 18 is a drawing for explaining a process performed before arrival of a patient according to a third embodiment;

FIG. 19 is a block diagram of a medical information management system according to the third embodiment;

FIG. 20 is a drawing illustrating an example of an image table according to the third embodiment;

FIG. 21 is a sequence chart illustrating an example of the process performed before the arrival of the patient according to the third embodiment;

FIG. 22 is a flowchart illustrating an example of an image data obtaining process according to the third embodiment;

FIG. 23 is a drawing for explaining a process performed before arrival of a patient according to a fourth embodiment;

FIG. 24 is a drawing illustrating an example of medical information data according to the fourth embodiment;

FIG. 25 is a block diagram of a medical information management system according to the fourth embodiment;

FIG. 26 is a drawing illustrating an example of a patient table according to the fourth embodiment;

FIG. 27 is a drawing illustrating an example of an application log according to the fourth embodiment;

FIG. 28 is a drawing illustrating an example of a device table according to the fourth embodiment;

FIG. 29A is a drawing illustrating an example of an image table according to the fourth embodiment;

FIG. 29B is a drawing illustrating another example of the image table according to the fourth embodiment;

FIG. 30 is a sequence chart illustrating an example of a medical information obtaining process according to the fourth embodiment; and

FIG. 31 is a flowchart illustrating an example of a medical information updating process according to the fourth embodiment.

### DETAILED DESCRIPTION

A medical information management system according to an embodiment includes a storage device and a medical information management apparatus. The storage device stores therein pieces of medical information of patients acquired by an extra-order device that acquires medical information without following a medical order issued by a hospital. The medical information management apparatus is configured to obtain medical information of a patient who is to use the hospital, from among the pieces of medical information stored in the storage device. The medical information management apparatus includes a processing circuit configured: to obtain the medical information of the patient who is to use the hospital, from among the pieces of medical information stored in the storage device; to obtain patient identification information uniquely identifying, within the hospital, the patient who is to use the hospital, and to further bring the obtained medical information of the patient into association with the patient identification information.

Exemplary embodiments of a medical information management system, a medical information management apparatus, and a medical information management method will be explained below, with reference to the accompanying drawings. Possible embodiments are not limited to the embodiments described below. Further, the description of each of the embodiments is, in principle, similarly applicable to any other embodiment.

A medical information management system according to an embodiment is configured to bring medical information such as one or more image of a patient acquired from an ambulance or the like governed by a fire department or a firefighters organization, into association with medical information held at a hospital or the like being the transport destination of the patient.

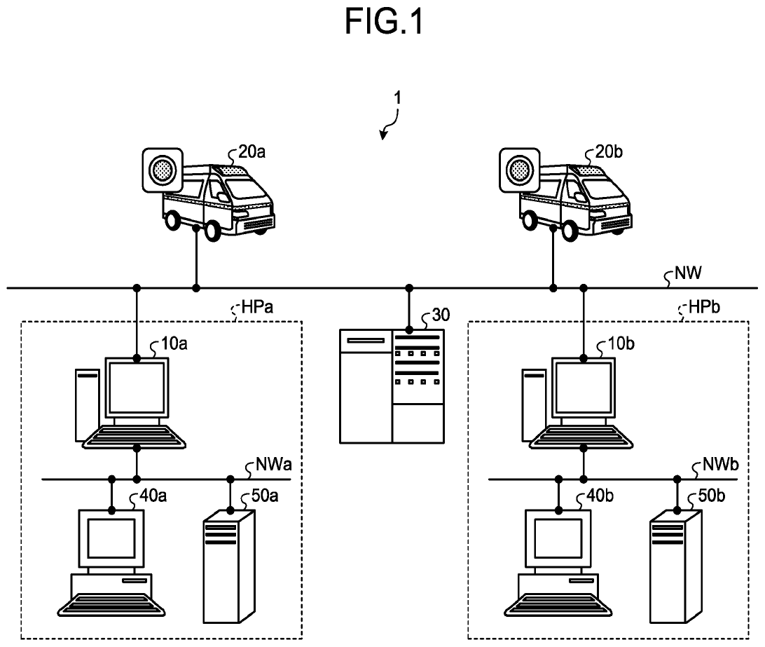

FIG. 1 is a diagram illustrating an exemplary configuration of a medical information management system. As illustrated in FIG. 1, a medical information management system 1 according to the present embodiment includes management apparatuses 10a and 10b, onboard devices 20a and 20b, an image server 30, Radiology Information Systems (RISs) 40a and 40b, and Picture Archiving and Communication Systems (PACSs) 50a and 50b.

In the medical information management system 1, the management apparatuses 10a and 10b, the onboard devices 20a and 20b, and the image server 30 are connected so as to be able to communicate with one another via a network NW formed in a wired or wireless manner. Further, the management apparatus 10a, the RIS 40a, and the PACS 50a are each connected via an intra-hospital network NWa formed in a wired or wireless manner, so as to be able to communicate with a hospital information system (not illustrated) structured in a hospital HPa. Similarly, the management apparatus 10b, the RIS 40b, and the PACS 50b are each connected via an intra-hospital network NWb formed in a wired or wireless manner, so as to be able to communicate with a hospital information system (not illustrated) structured in a hospital HPb. Also modalities (explained later) and server apparatuses (not illustrated) included in other systems used at the hospital such as a hospital accounting system (not illustrated), an electronic medical record system (not illustrated), and the like may be connected to the intra-hospital networks NWa and NWb.

The medical information management system 1 illustrated in FIG. 1 includes the two management apparatuses 10a and 10b and the two onboard devices 20a and 20b; however, the quantities of the apparatuses and the devices are arbitrary. For example, three or more management apparatuses may be connected to the network NW. In the following sections, when the management apparatuses 10a and 10b are not distinguished from each other, these apparatuses may each simply be referred to as a “management apparatus 10”. Similarly, the onboard devices 20a and 20b, the RISs 40a and 40b, the PACSs 50a and 50b, the hospitals HPa and HPb, and the intra-hospital networks NWa and NWb also may each be referred to as an “onboard device 20”, an “RIS 40”, a “PACS 50”, a “hospital HP”, and an “intra-hospital network NW”.

The management apparatuses 10 illustrated in FIG. 1 are, for example, computers located in the hospitals HPa and HPb. For example, the management apparatus 10a is configured to obtain medical information including one or more images of a patient during transport and being obtained prior to arrival at the hospital HPa, from the image server 30, and to bring the obtained medical information into association with information identifying the patient at the hospital HPa and being obtained from the RIS 40a. Further, the management apparatus 10a is configured to convert the obtained medical information into a Digital Imaging and Communications in Medicine (DICOM) format and to output the result to the PACS 50a. In the following sections, the medical information of the patient obtained before the arrival at the hospital may be referred to as “pre-arrival medical information”. The identifier used for uniquely identifying the patient at the hospital may be referred to as an “emergency identifier (ID)”.

The onboard devices 20 illustrated in FIG. 1 are each a device mounted in a transporter such as an ambulance that transports a patient, for example. Each of the onboard devices 20 is configured to take one or more images of the patient during transport and to transmit the pre-arrival medical information including the images and the like to the image server 30. Each of the onboard devices 20 is an example of a device provided outside the hospital (hereinafter “extra-order device”). Further, in the present embodiment, one onboard device 20 is mounted in each of the transporters (e.g., the ambulances), so that the transporters and the onboard devices 20 are in one-to-one correspondence.

The image server 30 illustrated in FIG. 1 is, for example, a computer installed at the fire department or the like. The image server 30 is configured to obtain the pre-arrival medical information including the images and the like from the onboard devices 20 or the like and to store the obtained pre-arrival medical information into a storage device. In response to a request from either of the management apparatuses 10a and 10b, for example, the image server 30 is configured to transmit the stored pre-arrival medical information. The image server 30 is an example of the storage device.

The RISs 40 illustrated in FIG. 1 are known computers installed in the hospitals HPa and HPb and are each configured to manage medical order (hereinafter, simply “order”) related to medical examinations and diagnosing processes performed at the hospital. When having received an input of a schedule to transport an emergency patient to the hospital HPa, for example, the RIS 40a is configured to issue an emergency ID as an identifier uniquely identifying the patient corresponding to the order and to output the issued emergency ID to the management apparatus 10a and to the PACS 50a.

The PACSs 50 illustrated in FIG. 1 are computers installed in the hospitals HPa and HPb, for example. Each of the PACSs 50 is a known computer configured to obtain the medical information in a DICOM format and to store the obtained medical information into a storage device. For example, the PACS 50a is configured to obtain medical information converted into the DICOM format from the management apparatus 10a and to store the obtained medical information into a storage device. Further, the PACS 50a is configured to obtain medical information including one or more images of a patient obtained after the arrival at the hospital HPa, from a modality such as Computed Radiography (CR), Computed Tomography (CT), Magnetic Resonance Imaging (MRI), or the like. The medical information of the patient obtained after the arrival at the hospital may be referred to as “post-arrival medical information”. Further, the pre-arrival medical information is an example of medical information acquired before issuance of an order by the hospital and acquired by an extra-order device such as the onboard devices 20 or the like, i.e., the medical information of the patient acquired by an extra-order device without following an order issued by the hospital. The post-arrival medical information is an example of the medical information of the patient obtained by following an order issued by the hospital.

### First Embodiment

To begin with, a first embodiment will be explained. In the first embodiment, the management apparatus 10a is configured to specify the pre-arrival medical information of the patient by using a “Device ID” uniquely identifying the onboard device 20a that took the images of the patient and information related to the arrival time of the patient at the hospital. In this situation, the “Device ID” is an example of the identification information of an extra-order device. The “arrival time” is an example of the arrival status. For example, the Device ID of the onboard device 20a is “Camera-A”, whereas the Device ID of the onboard device 20b is “Camera-B”. As explained below, in some situations, a Device ID may be assigned to each of devices other than the onboard devices 20.

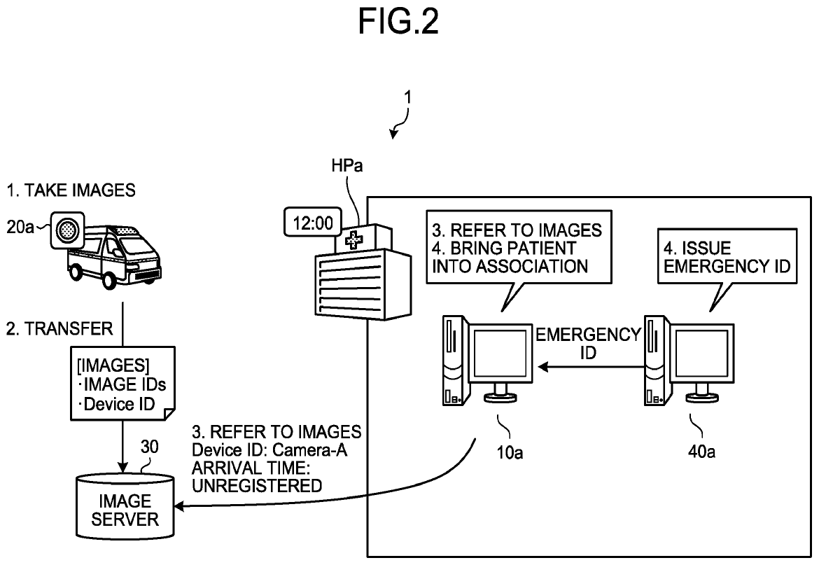

First, a process performed before the patient arrives at the hospital HPa will be explained. FIG. 2 is a drawing for explaining the process performed before the arrival of the patient according to the first embodiment. The sequence in which the processes in the drawings are performed is an example. It is possible to reverse the sequence for one or more of the processes. It is also acceptable to simultaneously perform two or more of the processes in parallel to one another.

As illustrated in FIG. 2, at first, the onboard device 20a mounted in the ambulance transporting the patient takes one or more images of the patient at time “12:00” and assigns “image IDs” each being an identifier uniquely identifying a different one of the images (1. Take Images). The onboard device 20a transmits the taken image to the image server 30, together with the “image IDs” and the “Device ID” (2. Transfer). The image server 30 stores therein the image transmitted thereto from the onboard device 20a, so as to be kept in correspondence with the image IDs and the Device ID.

Further, the hospital HPa is notified of a schedule of the transport of the patient in the ambulance, together with the Device ID of the onboard device 20a mounted in the ambulance. The transport schedule may be notified by the onboard device 20a, for example. However, possible embodiments are not limited to this example, and the schedule may be notified by the image server 30 or another device. In that situation, the management apparatus 10a at the hospital HPa refers to the images stored in the image server 30 (3. Refer to Images). At that time, among the images kept in association with the Device ID in the notification, the management apparatus 10a refers to such images that are not kept in association with an arrival time, i.e., images of which the arrival times are “unregistered”.

Subsequently, the RIS 40a issues an “emergency ID” used for identifying the patient at the hospital HPa and transmits the “emergency ID” to the management apparatus 10a (4. Issue Emergency ID). The management apparatus 10a brings the obtained emergency ID into association with the Device ID and the arrival time (4. Bring Patient into Association). In this situation, the “emergency ID” is an example of the patient identification information. The processes of “3. Refer to Images” and “4. Issue Emergency ID” with “4. Bring Patient into Association” may simultaneously be performed in parallel to each other or may be performed in a reversed sequence.

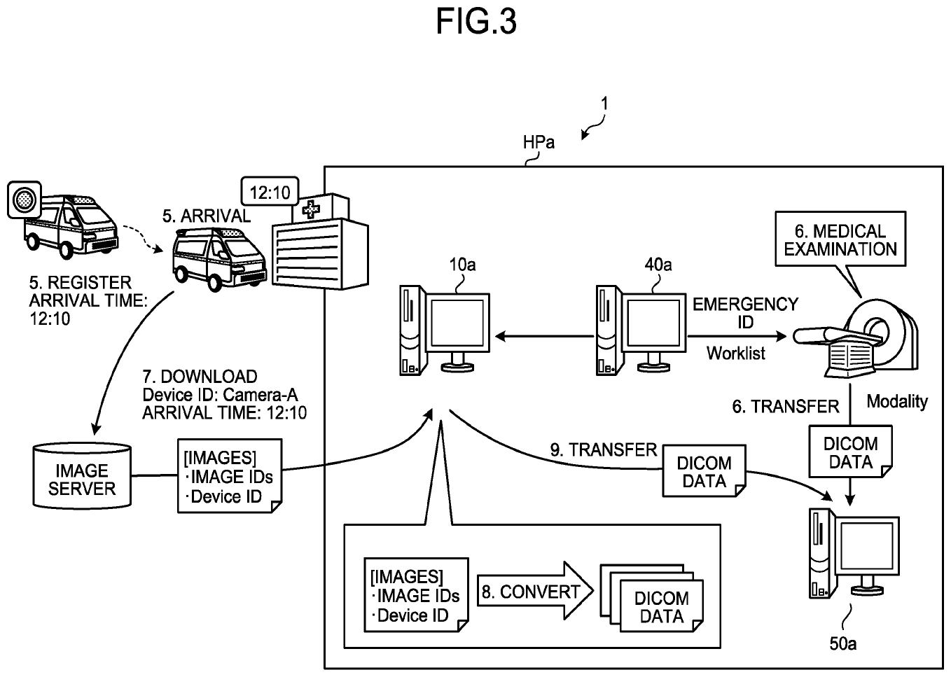

Next, a process performed after the patient arrives at the hospital HPa will be explained. FIG. 3 is a drawing for explaining the process performed after the arrival of the patient according to the first embodiment. As illustrated in FIG. 3, the patient transported by the ambulance in which the onboard device 20a is mounted arrives at the hospital HPa at time “12:10” (5. Arrival). In this situation, the onboard device 20a transmits the arrival time “12:10” to the image server 30 (5. Register).

Further, at the hospital HPa, a medical examination is performed on the patient who has arrived, by using a modality for which the already-issued emergency ID and a worklist are provided by the RIS 40a in advance (6. Medical Examination). One or more images of the patient obtained during the medical examination are transmitted to the PACS 50a, as data in the DICOM format kept in association with the emergency ID (6. Transfer). The PACS 50a stores therein the obtained data in the DICOM format as post-arrival medical information.

Further, when the patient arrives, the management apparatus 10a downloads the pre-arrival medical information including the images of the patient, from the image server 30 (7. Download). In this situation, among the images kept in association with the Device ID provided in the notification in advance, the management apparatus 10a downloads such images of which the arrival times coincide with the arrival time of the patient, i.e., the images of which the arrival times are each “12:10”.

Subsequently, the management apparatus 10a converts the downloaded images into data in the DICOM format, together with the image IDs, emergency ID, and Device ID that are kept in association (8. Convert), and transmits the result to the PACS 50a, as pre-arrival medical information (9. Transfer). The PACS 50a stores therein the received pre-arrival medical information converted in the DICOM format. As a result, in the PACS 50a, the pre-arrival medical information and the post-arrival medical information are brought into association with each other by the same emergency ID.

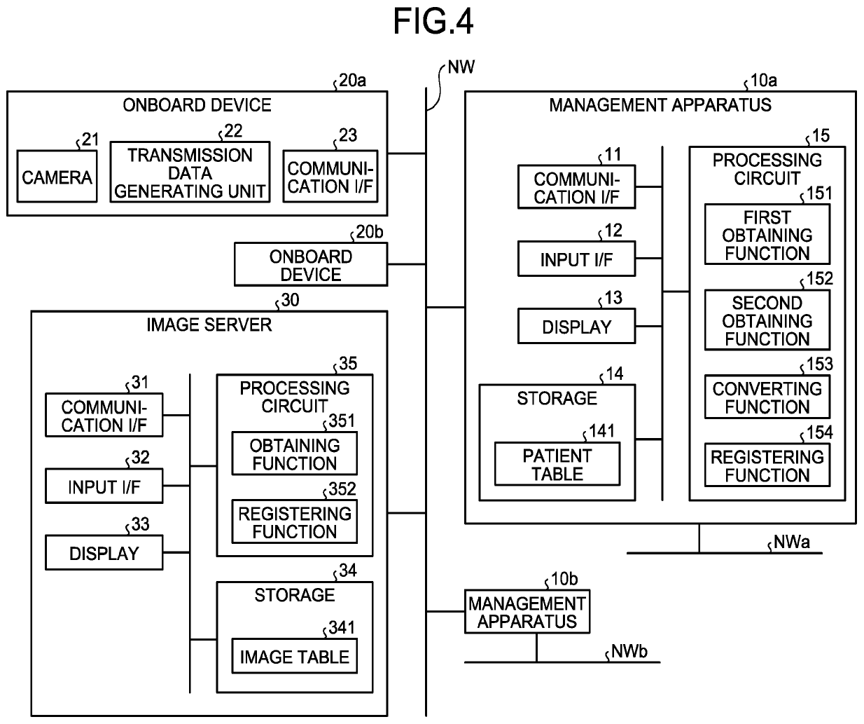

Next, a function of the medical information management system 1 that performs the processes illustrated in FIGS. 2 and 3 will be explained. FIG. 4 is a block diagram of a medical information management system according to the first embodiment. As illustrated in FIG. 4, the management apparatus 10a connected to the intra-hospital network NWa of the hospital HPa includes a communication interface (I/F) 11, an input I/F 12, a display 13, a storage 14, and a processing circuit 15. In this situation, because the management apparatus 10b connected to the intra-hospital network NWb of the hospital HPb has the same configuration as that of the management apparatus 10a, the management apparatuses 10a and 10b may each be referred to as a management apparatus 10 hereinafter, without being distinguished from each other.

The communication I/F 11 is connected to the processing circuit 15 and is configured to control transmission and communication of various types of data, to/from and with various types of apparatuses and devices connected via the network NW and the intra-hospital network NWa. For example, the communication I/F 11 may be realized by using a network card, a network adaptor, a Network interface Controller (NIC), or the like.

The input I/F 12 is connected to the processing circuit 15 and is configured to convert an input operation received from an administrator (not illustrated) of the management apparatus 10 into an electrical signal and to output the electrical signal to the processing circuit 15. For example, the input I/F 12 may be a switch button, a mouse, a keyboard, a touch panel, and/or the like.

The display 13 is connected to the processing circuit 15 and is configured to display various types of information and various types of image data output from the processing circuit 15. For example, the display 13 may be realized by using a liquid crystal monitor, a Cathode Ray Tube (CRT) monitor, a touch panel, or the like.

The storage 14 is connected to the processing circuit 15 and is configured to store therein various types of data. For example, the storage 14 may be realized by using a semiconductor memory element such as a Random Access Memory (RAM) or a flash memory, or a hard disk, an optical disk, or the like. In the present embodiment, the storage 14 stores therein a patient table 141, for example.

The patient table 141 stores therein information keeping the pre-arrival medical information obtained from the image server 30 in correspondence with the emergency ID obtained from the RIS 40. In the present embodiment, for example, the patient table 141 stores therein information related to the images included in the pre-arrival medical information. The information stored in the patient table 141 is stored by, for example, a first obtaining function 151, a second obtaining function 152, a converting function 153, and a registering function 154, (explained later).

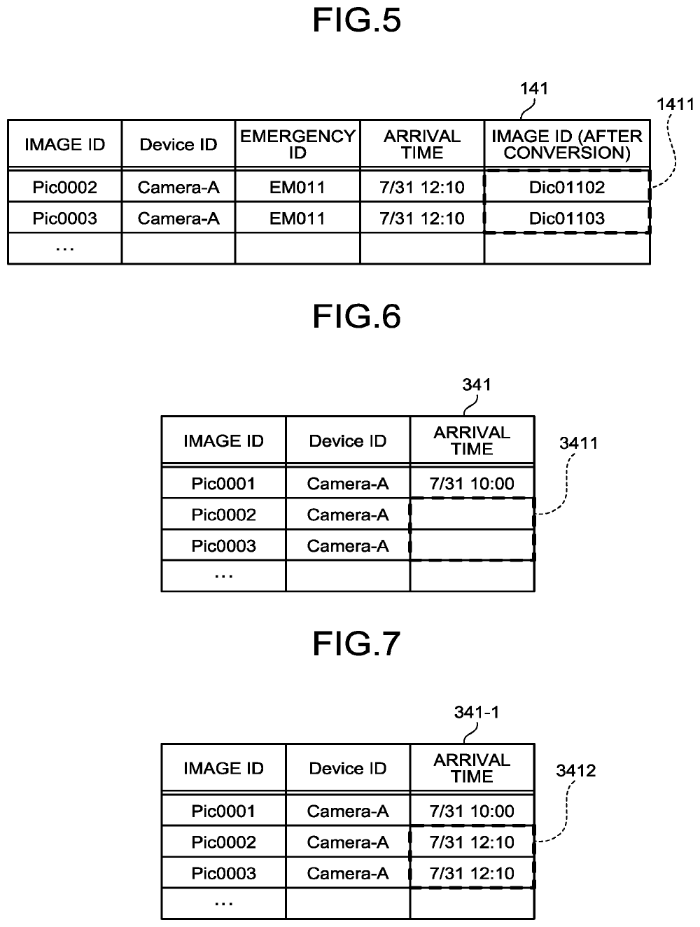

FIG. 5 is a drawing illustrating an example of the patient table according to the first embodiment. For example, as illustrated in FIG. 5, the patient table 141 is information keeping “image IDs”, “Device IDs”, “emergency IDs”, “arrival times”, and “image IDs (after conversion)” in correspondence with one another. In FIG. 5, the “image IDs” are each an identifier uniquely identifying an image included in the pre-arrival medical information. The “Device IDs” are each an identifier uniquely identifying the onboard device 20 that obtained the image. The “emergency IDs” are each an emergency ID obtained from the RIS 40. The “arrival times” each indicate the time at which the patient specified by the emergency ID arrived at the hospital. The “image IDs (after conversion)” are each an identifier uniquely identifying an image converted into the DICOM format and specified by the image ID.

For example, the record in the first line in FIG. 5 indicates that the pre-arrival medical information including the image identified with the image ID “Pic0002” was obtained by the onboard device identified with the Device ID “Camera-A” and has the emergency ID “EMO11” assigned thereto. Further, the record in the first line in FIG. 5 indicates that the patient specified by the emergency ID arrived at the hospital “on 7/31 at 12:10”. Further, the record in the first line in FIG. 5 indicates that the image identified with the image ID “Pic0002” was converted into the image identified with the image ID “Dic01102”. Similarly, the record in the second line in FIG. 5 indicates that the pre-arrival medical information including the image identified with the image ID “Pic00003” was also obtained by the onboard device identified with the Device ID “Camera-A” and has the emergency ID “EM011” assigned thereto, similarly to the pre-arrival medical information in the record in the first line. In other words, because the record in the second line in FIG. 5 is a record related to the pre-arrival medical information of the same patient as the record in the first line, the arrival time is also the same as that in the record in the first line. Further, the record in the second line in FIG. 5 indicates that the image identified with the image ID “Pic0003” was converted into the image identified with the image ID “Dic01103”.

In FIG. 5, unregistered items in the records are left blank, for example. For instance, when the patient specified by the Device ID “Camera-A” and the emergency ID “EM011” has not yet arrived at the hospital, the item of the arrival time remains blank. Further, for example, if the images had not yet been converted into the DICOM format, an item 1411 indicating the image IDs (after conversion) would remain blank.

Returning to the description of FIG. 4, the processing circuit 15 is configured to control operations of the entirety of the management apparatus 10, by executing the first obtaining function 151, the second obtaining function 152, the converting function 153, and the registering function 154. In this situation, the first obtaining function 151 is an example of a first obtaining unit. The second obtaining function 152 is an example of a second obtaining unit. The converting function 153 is an example of a converting unit. The registering function 154 is an example of an associating unit. In this situation, for example, processing functions of the constituent elements of the processing circuit 15 illustrated in FIG. 4, namely, the first obtaining function 151, the second obtaining function 152, the converting function 153, and the registering function 154, are recorded in the storage 14 in the form of computer-executable programs. The processing circuit 15 is configured to realize the functions corresponding to the programs, by reading the programs from the storage 14 and executing the read programs. In other words, the processing circuit 15 that has read the programs has the functions illustrated within the processing circuit 15 in FIG. 4.

In this situation, all the processing functions of the first obtaining function 151, the second obtaining function 152, the converting function 153, and the registering function 154 may be recorded in the storage 14 in the form of one computer-executable program. For example, the program may be referred to as a medical information management program. In that situation, the processing circuit 15 realizes the first obtaining function 151, the second obtaining function 152, the converting function 153, and the registering function 154 corresponding to the medical information management program, by reading the medical information management program from the storage 14 and executing the read medical information management program.

The first obtaining function 151 included in the processing circuit 15 is configured to obtain the pre-arrival medical information from the image server 30. For example, by using the input of the Device ID provided in the notification or the like as a trigger of the start, the first obtaining function 151 is configured to access the image server 30 via the network NW. The first obtaining function 151 is configured to refer to the images that are stored in the image server 30 and are kept in association with the Device ID. Further, the first obtaining function 151 is configured to output the image IDs of the obtained images and the arrival times kept in association with the images, to the registering function 154.

Further, by using the input of the notification about the arrival of the patient or the like as a trigger of the start, the first obtaining function 151 is configured to download the image data from the image server 30. The first obtaining function 151 is configured to download the pre-arrival medical information including the data of the images corresponding to the image IDs and the Device ID that have already been registered in the patient table 141 and to further output the downloaded pre-arrival medical information to the converting function 153.

The second obtaining function 152 is configured to obtain the emergency ID from the RIS 40a. For example, the second obtaining function 152 is configured to receive the emergency ID from the RIS 40a and to output the received emergency ID to the registering function 154.

The converting function 153 is configured to convert the image data into data in the DICOM format. When having received the pre-arrival medical information including the data of the images that is output from the first obtaining function 151, the converting function 153 is configured to convert the images into data in the DICOM format, together with the image IDs, Device ID, and emergency ID kept in association therewith. The converting function 153 is configured to transmit the pre-arrival medical information resulting from the conversion, to the PACS 50a.

The registering function 154 is configured to bring the pre-arrival medical information of the transported patient into correspondence with the emergency ID. When having received the image IDs and the arrival time that is output from the first obtaining function 151, the registering function 154 is configured to judge whether an “arrival time” has not yet been registered (i.e., is unregistered) with respect to pre-arrival medical information having a matching Device ID. The registering function 154 is configured to store the image IDs and the Device ID of the pre-arrival medical information of which the arrival time is determined to be unregistered, into the patient table 141 so as to be kept in association with the emergency ID output from the second obtaining function 152.

Further, each of the onboard devices 20 includes, as illustrated in FIG. 4, a camera 21, a transmission data generating unit 22, and a communication I/F 23. Because the onboard devices 20a and 20b have the same configuration as each other, the onboard devices 20a and 20b may each be referred to as an onboard device 20 hereinafter, without being distinguished from each other.

The camera 21 included in the onboard device 20 is configured to obtain the one or more images of the patient and to output data of the images to the transmission data generating unit 22. The camera 21 may be an optical camera, for example. However, possible embodiments are not limited to this example, and may be another device such as an X-ray CT apparatus, an ultrasound diagnosis apparatus, or the like. The transmission data generating unit 22 is configured to generate the pre-arrival medical information to be transmitted to the image server 30, by assigning the identification information to the data of the images. The transmission data generating unit 22 is configured to issue image IDs for the data of the images, to assign the issued image IDs to the data of the images together with the Device ID assigned to the onboard device 20, and to transmit the result to the image server 30 via the communication I/F 23. Further, the transmission data generating unit 22 is configured to transmit the transport schedule of the patient including the Device ID to the hospital HPa via the communication I/F 23.

The communication I/F 23 is configured to control transmission and communication of various types of data, to/from and with other apparatuses and devices or the like connected via the network NW. For example, the communication I/F 23 is configured to transmit the pre-arrival medical information output from the transmission data generating unit 22, to the image server 30. For example, the communication I/F 23 may be realized by using a network card, a network adaptor, or the like.

As illustrated in FIG. 4, the image server 30 includes a communication I/F 31, an input I/F 32, a display 33, a storage 34, and a processing circuit 35.

The communication I/F 31 is connected to the processing circuit 35 and is configured to control transmission and communication of various types of data, to/from and with various types of apparatuses and devices connected via the network NW. For example, the communication I/F 31 may be realized by using a network card, a network adaptor, or the like.

The input I/F 32 is connected to the processing circuit 35 and is configured to convert an input operation received from an administrator (not illustrated) of the image server 30 into an electrical signal and to output the electrical signal to the processing circuit 35. Fr example, the input I/F 32 may be a switch button, a mouse, a keyboard, a touch panel, and/or the like.

The display 33 is connected to the processing circuit 35 and is configured to display various types of information output from the processing circuit 35 and the like. For example, the display 33 may be realized by using a liquid crystal monitor, a CRT monitor, a touch panel, or the like.

The storage 34 is connected to the processing circuit 35 and is configured to store therein various types of data. For example, the storage 34 may be realized by using a semiconductor memory element such as a RAM or a flash memory, or a hard disk, an optical disk, or the like. In the present embodiment, the storage 34 stores therein an image table 341, for example.

The image table 341 stores therein information related to the pre-arrival medical information obtained from the onboard device 20. In the present embodiment, the image table 341 is set, for example, by an obtaining function 351 and a registering function 352. For example, as illustrated in FIG. 6, the image table 341 stores therein “image IDs”, “Device IDs”, and “arrival times” so as to be kept in correspondence with one another.

FIG. 6 is a drawing illustrating an example of the image table according to the first embodiment. Because the items in FIG. 6 are similar to the items illustrated in FIG. 5, detailed explanations thereof will be omitted. In the example in FIG. 6, arrival times 3411 corresponding to the image IDs “Pic0002” and “Pic0003” and the Device ID “Camera-A” are unregistered. In other words, the image table 341 in FIG. 6 indicates that the patient corresponding to the image IDs and the Device ID has not yet arrived at the hospital.

Returning to the description of FIG. 4, the processing circuit 35 is configured to control operations of the entirety of the image server 30, by executing the obtaining function 351 and the registering function 352. In this situation, for example, processing functions of the constituent elements of the processing circuit 35 illustrated in FIG. 4, namely, the obtaining function 351 and the registering function 352 are recorded in the storage 34 in the form of computer-executable programs. The processing circuit 35 is configured to realize the functions corresponding to the programs, by reading the programs from the storage 34 and executing the read programs. In other words, the processing circuit 35 that has read the programs has the functions illustrated within the processing circuit 35 in FIG. 4.

In this situation, all the processing functions of the obtaining function 351 and the registering function 352 may be recorded in the storage 34 in the form of one computer-executable program. In that situation, the processing circuit 35 realizes the obtaining function 351 and the registering function 352 by reading the program from the storage 34 and executing the read program.

The obtaining function 351 included in the processing circuit 35 is configured to obtain the pre-arrival medical information from the onboard device 20. For example, when having received the pre-arrival medical information including the data of the images from the onboard device 20 via the network NW, the obtaining function 351 stores the data of the images into the storage 34 and also outputs information such as the image IDs and the Device ID kept in association with the pre-arrival medical information, to the registering function 352.

The registering function 352 is configured to register information related to the pre-arrival medical information into the image table 341. The registering function 352 is configured to register the image IDs and the Device ID output from the obtaining function 351 into the image table 341 illustrated in FIG. 6. Further, when having obtained the information related to the arrival time from the onboard device 20, the registering function 352 stores the information related to the arrival time so as to be kept in association with the image IDs and the Device ID stored in the image table 341, as illustrated in FIG. V.

FIG. 7 is a drawing illustrating another example of the image table according to the first embodiment. In items 3412 for “arrival times” illustrated in FIG. 7, “7/31, 12:10” is registered in the boxes for the arrival times, which were blank in FIG. 6. In other words, another example of the image table 341-1 illustrated in FIG. 7 indicates that the patient corresponding to the image IDs “Pic0002” and “Pic0003” and the Device ID “Camera-A” arrived at the hospital “on 7/31 at 12:10”.

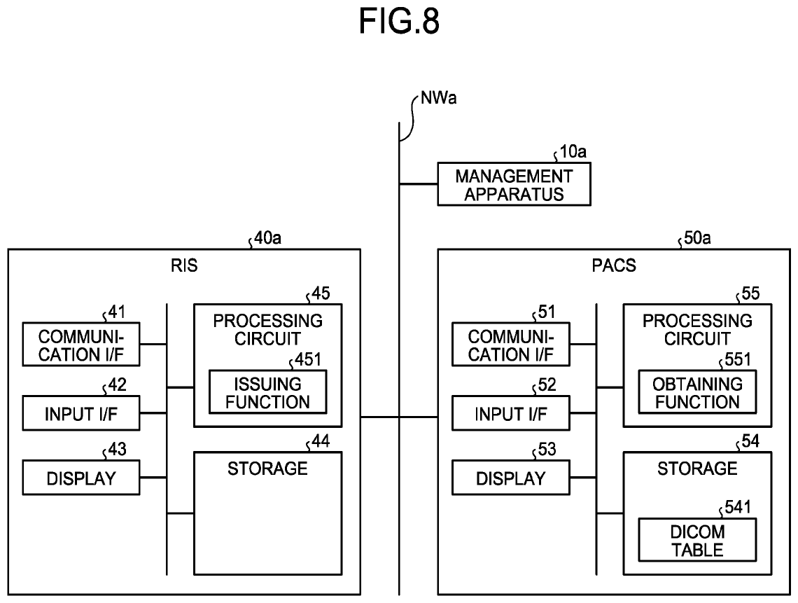

Next, a function of the medical information management system 1 that performs the processes illustrated in FIG. 3 will be explained. FIG. 8 is a block diagram illustrating a part of the hospital information system according to the first embodiment. As illustrated in FIG. 8, to the intra-hospital network NWa at the hospital HPa included in the medical information management system 1, the RIS 40a and the PACS 50a are communicably connected, in addition to the management apparatus 10a is. Also, to the intra-hospital network NWb at the hospital HPb, the RIS 40b and the PACS 50b are similarly connected. Accordingly, in the following sections, the RISs 40a and 40b and the PACS 50a and 50b may not be distinguished from each other.

As illustrated in FIG. 8, the RIS 40a includes a communication I/F 41, an input I/F 42, a display 43, a storage 44, and a processing circuit 45.

The communication I/F 41 is connected to the processing circuit 45 and is configured to control transmission and communication of various types of data, to/from and with various types of apparatuses and devices connected via the intra-hospital network NWa. For example, the communication I/F 41 may be realized by using a network card, a network adaptor, or the like.

The input I/F 42 is connected to the processing circuit 45 and is configured to convert an input operation received from an administrator (not illustrated) of the RIS 40a into an electrical signal and to output the electrical signal to the processing circuit 45. For example, the input I/F 42 may be a switch button, a mouse, a keyboard, a touch panel, and/or the like.

The display 43 is connected to the processing circuit 45 and is configured to display various types of information output from the processing circuit 45 and the like. For example, the display 43 may be realized by using a liquid crystal monitor, a CRT monitor, a touch panel, or the like.

The storage 44 is connected to the processing circuit 45 and is configured to store therein various types of data. For example, the storage 44 may be realized by using a semiconductor memory element such as a RAM or a flash memory, or a hard disk, an optical disk, or the like.

The processing circuit 45 is configured to control operations of the entirety of the RIS 40a, by executing an issuing function 451. In this situation, for example, the issuing function 451, which is a constituent element of the processing circuit 45 illustrated in FIG. 8, is recorded in the storage 44 in the form of computer-executable programs. The processing circuit 45 is configured to realize the functions corresponding to the programs, by reading the programs from the storage 44 and executing the read programs. In other words, the processing circuit 45 that has read the programs has the functions illustrated within the processing circuit 45 in FIG. 4.

Alternatively, all the processing functions of the issuing function 451 may be recorded in the storage 44 in the form of one computer-executable program. In that situation, the processing circuit 45 realizes the issuing function 451, by reading the program from the storage 44 and executing the read program.

The issuing function 451 included in the processing circuit 45 is configured to issue the emergency ID. For example, by using the input of the Device ID provided in the notification or the like as a trigger of the start, the issuing function 451 is configured to issue the emergency ID and to output the emergency ID to the management apparatus 10a and to the PACS 50a via the intra-hospital network NWa.

A registering function 452 is configured to register information related to the pre-arrival medical information into an image table 441. The registering function 452 is configured to register the image IDs and the Device ID output from the issuing function 451 into the image table 441. Further, when having obtained the information related to the arrival time from the onboard device 20, the registering function 452 stores the information related to the arrival time so as to be kept in association with the image IDS and the Device ID stored in the image table 441, as illustrated in FIG. 7.

As illustrated in FIG. 8, the PACS 50a includes a communication I/F 51, an input I/F 52, a display 53, a storage 54, and a processing circuit 55.

The communication I/F 51 is connected to the processing circuit 55 and is configured to control transmission and communication of various types of data, to/from and with various types of apparatuses and devices connected via the intra-hospital network NWa. For example, the communication I/F 51 may be realized by using a network card, a network adaptor, or the like.

The input I/F 52 is connected to the processing circuit 55 and is configured to convert an input operation received from an administrator (not illustrated) of the PACS 50a into an electrical signal and to output the electrical signal to the processing circuit 55. For example, the input I/F 52 may be a switch button, a mouse, a keyboard, a touch panel, and/or the like.

The display 53 is connected to the processing circuit 55 and is configured to display various types of information output from the processing circuit 55 and the like. For example, the display 53 may be realized by using a liquid crystal monitor, a CRT monitor, a touch panel, or the like.

The storage 54 is connected to the processing circuit 55 and is configured to store therein various types of data. For example, the storage 54 may be realized by using a semiconductor memory element such as a RAM or a flash memory, or a hard disk, an optical disk, or the like. In the present embodiment, the storage 54 stores therein a DICOM table 541, for example.

The DICOM table 541 stores therein information related to data in the DICOM format. In the present embodiment, the DICOM table 541 is set by an obtaining function 551 (explained later), for example. The DICOM table 541 is an example of a storage unit.

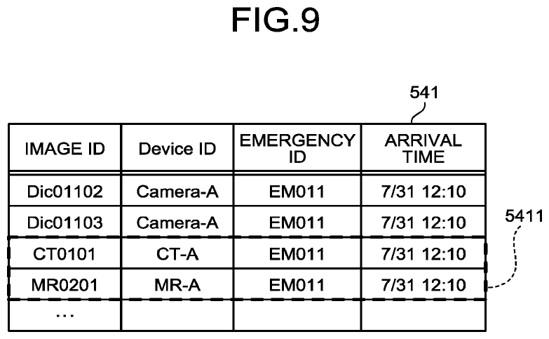

FIG. 9 is a drawing illustrating an example of the DICOM table according to the first embodiment. For example, as illustrated in FIG. 9, the DICOM table 541 stores therein “image IDs”, “Device IDs”, “emergency IDs” and “arrival times” so as to be kept in correspondence with one another. For example, the record in the first line in FIG. 9 indicates that the image identified with the image ID “Dic01102” was obtained by the onboard device identified with the Device ID “Camera-A” and has the emergency ID “EM011” assigned thereto. Further, the record in the first line in FIG. 9 indicates that the patient specified by the emergency ID arrived at the hospital “on 7/31 at 12:10”. The same applies to the image identified with the image ID “Dic01103” and indicated in the record in the second line in FIG. 9. These images were received as outputs of the management apparatus 10a and were converted into data in the DICOM format. For example, it is possible to specify these images by using the image IDs stored as the item 1411 of the image IDs (after conversion) in the patient table 141 illustrated in FIG. 5.

Further, as indicated in records 5411 in the third line and the fourth line in FIG. 9, the DICOM table 541 stores therein information related to images obtained from modalities other than the management apparatus 10a. For example, the record in the third line in FIG. 9 indicates that, in association with an image ID “CT0101” obtained by a modality (not illustrated) identified with a Device ID “CT-A”, the same emergency ID and the same arrival time as those of the image identified with the image ID “Dic01102” and the image identified with the image ID “Dic01103” are registered. Also, the record in the fourth line in FIG. 9 indicates that, in association with an image ID “MR0201” obtained by another modality (not illustrated) identified with a Device ID “MR-A”, the same emergency ID and the same arrival time are registered. In other words, the DICOM table 541 in FIG. 9 stores therein information indicating that the image identified with the image ID “Dic01102” and the image identified with the image ID “Dic01103” obtained from the management apparatus 10a and the image identified with the image ID “CT0101” and the image identified with the image ID “MR0201” obtained from the modalities are related to the same transported patient.

Returning to the description of FIG. 8, the processing circuit 55 is configured to control operations of the entirety of the PACS 50a by executing the obtaining function 551. In this situation, for example, the obtaining function 551, which is a constituent element of the processing circuit 55 illustrated in FIG. 8, is recorded in the storage 54 in the form of computer-executable programs. The processing circuit 55 is configured to realize the functions corresponding to the programs, by reading the programs from the storage 54 and executing the read programs. In other words, the processing circuit 55 that has read the programs has the functions illustrated within the processing circuit 55 in FIG. 8.

Alternatively, all the processing functions of the obtaining function 551 may be recorded in the storage 54 in the form of one computer-executable program. In that situation, the processing circuit 55 realizes the obtaining function 551, by reading the program from the storage 54 and executing the read program.

The obtaining function 551 included in the processing circuit 55 is configured to obtain the image data and to also store information related to the image data into the DICOM table 541. For example, the obtaining function 551 is configured to obtain the image data converted into the DICOM format from the management apparatus 10a, via the intra-hospital network NWa. Similarly, for example, the obtaining function 551 is configured to obtain the image data in the DICOM format from modalities via the intra-hospital network NWa. Further, the obtaining function 551 is configured to store the image IDs, the Device IDs, the emergency IDs, and the arrival times kept in association with the obtained image data, into the DICOM table 541.

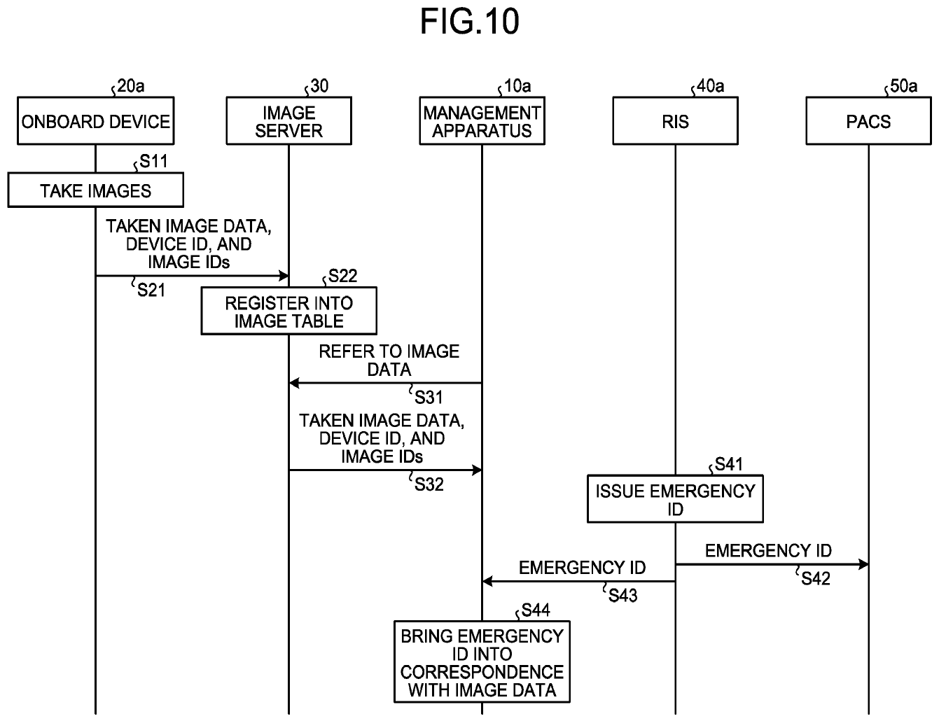

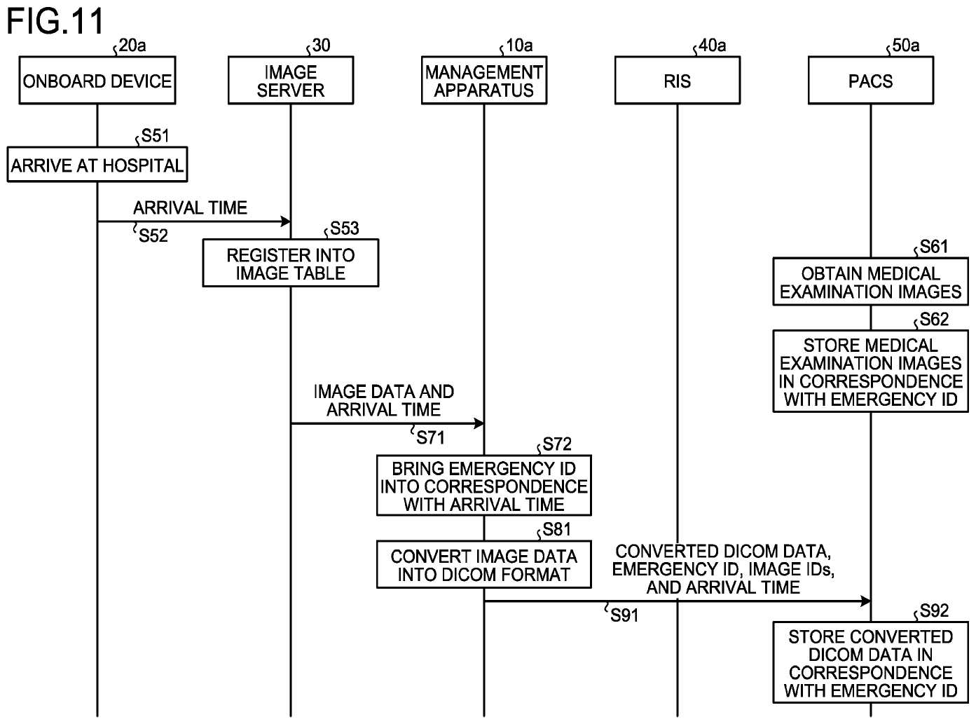

Next, a processing procedure performed by the medical information management system 1 according to the first embodiment will be explained, with reference to FIGS. 10 and 11. FIG. 10 is a sequence chart illustrating an example of the process performed before the arrival of the patient according to the first embodiment. In this situation, steps S31 and S32 in FIG. 10 are realized, for example, as a result of the processing circuit 15 included in the management apparatus 10a reading and executing the program corresponding to the first obtaining function 151 from the storage 14. Further, step S43 is realized, for example, as a result of the processing circuit 15 reading and executing the program corresponding to the second obtaining function 152 from the storage 14. Also, step S44 is realized, for example, as a result of the processing circuit 15 reading and executing the program corresponding to the registering function 154 from the storage 14.

For example, as illustrated in FIG. 10, in the medical information management system 1 according to the present embodiment, the onboard device 20a at first takes images of the patient during the transport, by using the camera 21 (step S11). The onboard device 20a transmits the taken image data to the image server 30, so as to be kept in association with the image IDs and the device (step S21).

Subsequently, when having obtained the image data, the image IDs, and the Device ID, the image server 30 registers the image IDs and the Device ID into the image table 341 (step S22). After that, when having obtained the Device ID, the processing circuit 15 refers to the images stored in the image server 30 by using the Device ID (step S31). The processing circuit 15 obtains the data and the image IDs of the images corresponding to the Device ID (step S32).

Further, when having obtained the Device ID, the RIS 40a issues an emergency ID used for specifying the patient being transport (step S41). The RIS 40a transmits the issued emergency ID to the PACS 50a (step S42) and also to the management apparatus 10a (step S43). The processing circuit 15 stores the emergency ID obtained from the RIS 40a so as to be kept in correspondence with the image data obtained from the image server 30 (step S44).

Next, a process performed in the medical information management system 1 after the arrival of the patient will be explained. FIG. 11 is a sequence chart illustrating an example of the process performed after the arrival of the patient according to the first embodiment. In this situation, step S71 in FIG. 11 is realized, for example, as a result of the processing circuit 15 included in the management apparatus 10a reading and executing the program corresponding to the first obtaining function 151 from the storage 14. Further, step S72 is realized, for example, as a result of the processing circuit 15 reading and executing the program corresponding to the registering function 154 from the storage 14. Also, steps S81 and S91 are realized, for example, as a result of the processing circuit 15 reading and executing the program corresponding to the converting function 153 from the storage 14.

At first, when the patient arrives at the hospital (step S51), the onboard device 20a transmits the arrival time to the image server 30 (step S52). The image server 30 registers the received arrival time into the image table 341 so as to be kept in correspondence with the Device ID and the image IDs (step S53).

Further, the PACS 50a obtains medical examination images of the patient who arrived at the hospital, from other modalities (step S61). The PACS 50a registers the obtained medical examination images into the DICOM table 541 so as to be kept in correspondence with the emergency ID (step S62).

The processing circuit 15 accesses the image server 30 and downloads the image data newly registered with the arrival time, together with the arrival time (step S71). Subsequently, the processing circuit 15 registers the obtained arrival time into the patient table 141 so as to be kept in correspondence with the emergency ID (step S72). Further, the processing circuit 15 converts the obtained image data into the DICOM format (step S81) and transmits the DICOM data resulting from the conversion to the PACS 50a so as to be kept in association with the emergency ID (step S91). The PACS 50a stores the DICOM data received from the management apparatus 10a into the DICOM table 541 so as to be kept in correspondence with the emergency ID (step S92).

As explained above, according to the first embodiment, the first obtaining function 151 is configured to obtain the pre-arrival medical information, which is the medical information of the patient obtained before the patient arrives at the hospital. The second obtaining function 152 is configured to obtain the patient identification information used for identifying the patient at the hospital and kept in correspondence with the post-arrival medical information, which is the medical information obtained after the patient arrives at the hospital. Further, the registering function 154 is configured to bring the pre-arrival medical information and the patient identification information into association with each other. Accordingly, the medical information management system 1 according to the first embodiment is able to easily bring the data related to the patient and acquired before the arrival at the hospital into association, at the hospital being the transport destination.

In that situation, as the information brought into association with the pre-arrival medical information, the first obtaining function 151 may further obtain the arrival information related to the arrival of the patient at the hospital. Further, the registering function 154 may bring pre-arrival medical information that is not kept in association with the arrival information indicating the arrival of the patient at the hospital, into association with the patient identification information. As a result, it is possible to prevent misidentification with information related to another patient who has already arrived at the hospital. The arrival information is an example of the arrival status.

Further, in the first embodiment, the medical information management system 1 includes the storage device (the image server 30) and the management apparatus 10. The storage device stores therein the pieces of medical information of patients acquired by an extra-order device that acquires medical information without following an order issued by the hospital HP. The processing circuit 15 included in the medical information management apparatus is configured to obtain the medical information of the patient who is to use the hospital, from among the pieces of medical information stored in the storage device, to obtain the patient identification information (the emergency ID) uniquely identifying, within the hospital, the patient who is to use the hospital, and to bring the obtained medical information of the patient into association with the patient identification information. With these arrangements, it is possible to easily bring the data related to the patient and acquired without following an order into association, at the hospital being the transport destination.

Further, in the first embodiment, the storage device may store therein the identification information (the Device ID) of the extra-order device, the arrival status at the hospital (the arrival time), and the medical information based on the extra-order device (the onboard device 20) so as to be kept in correspondence with one another. The processing circuit 15 included in the medical information management apparatus may obtain the identification information of the extra-order device and may obtain the medical information of the patient who is to use the hospital on the basis of the arrival status at the hospital kept in correspondence with the identification information.

Further, in the first embodiment, the patient identification information may be issued for an order issued by the hospital. The processing circuit 15 included in the medical information management apparatus may be configured to convert the obtained medical information of the patient into the same data format as the data format of the medical information that is based on an intra-hospital device used on the patient corresponding to the patient identification information and was obtained by following the issued order and configured to bring the medical information resulting from the conversion into association with the patient identification information.

The image IDs and the Device IDs in the first embodiment do not necessarily have to be able to uniquely identify the patients. For example, the image IDs do not necessarily have to contain information such as the patients' names or identification numbers for identifying the patients, and do not necessarily have to be kept in association with such information in advance.

With reference to FIG. 5, the example was explained in which the “arrival times” are stored as the arrival statuses; however, possible embodiments are not limited to this example. For instance, another arrangement is also acceptable in which the patient table 141 stores therein information indicating whether or not the patient has already arrived at the hospital. Further, yet another arrangement is also acceptable in which the patient table 141 or the image table 341 stores therein information indicating that the patient has not yet arrived at the hospital, so that the information is deleted upon arrival of the patient at the hospital. Further, the example was explained in which the onboard device 20a transmits the arrival time to the image server 30; however, possible embodiments are not limited to this example. Another arrangement is acceptable in which the onboard device 20a transmits only a notification of arrival, so that the image server 30 stores therein the time at which the notification of arrival is received as the arrival time. Further, the example was explained in which the arrival time is transmitted by the onboard device 20a to the image server 30; however, possible embodiments are not limited to this example. It is also acceptable to configure the management apparatus 10a to transmit the arrival time.

### Second Embodiment

In the first embodiment above, the configuration was explained in which the transported patient is specified by using the set made up of the Device ID of the onboard device 20a and the arrival time at which the patient arrived at the hospital. In contrast, in a second embodiment, a configuration will be explained in which a transported patient is specified by using a set made up of the Device ID of an onboard device 70a (explained later) and a transport ID uniquely identifying a patient transport procedure. In this situation, the “transport ID” is an example of the issued identification information. As the transport ID, for example, meta data appended on the basis of a publicly-known Encounter-Based Imaging Workflow (EBIW) may be used.

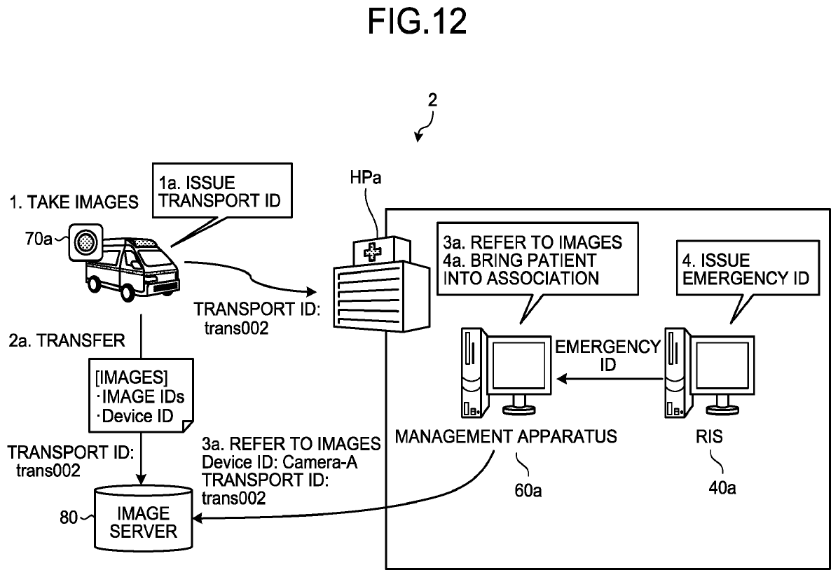

First, in the second embodiment, a process performed before the patient arrives at the hospital HPa illustrated in FIG. 1 will be explained. FIG. 12 is a drawing for explaining the process performed before the arrival of the patient according to the second embodiment. In the embodiments hereinafter, some of the elements that are the same as those illustrated in the drawings explained earlier will be referred to by using the same reference characters, and the duplicate explanations thereof will be omitted. Further, differences in the functions of a management apparatus 60a, the onboard device 70a, and an image server 80 illustrated in FIG. 12 according to the second embodiment, from the functions the management apparatus 10a, the onboard device 20a, and the image server 30 will individually be explained later.

As illustrated in FIG. 12, the onboard device 70a mounted in an ambulance transporting a patient takes one or more images of the patient and assigns “image IDs” thereto (1. Take Images). At this time, the onboard device 70a issues a transport ID “trans002” and transmits the transport ID to the hospital HPa (1a. Issue Transport ID).

Further, the onboard device 70a transmits the taken images to the image server 30, together with the “image IDs”, a “Device ID”, and the “transport ID” (2a. Transfer). The image server 30 stores therein the images transmitted thereto from the onboard device 20a, so as to be kept in correspondence with the image IDs, the Device ID, and the transport ID.

Further, the hospital HPa is notified of a schedule of the transport of the patient in the ambulance, together with the Device ID of the onboard device 70a mounted in the ambulance and the transport ID. In this situation, the management apparatus 60a at the hospital HPa refers to the images stored in the image server 80 (3a. Refer to Images). At that time, the management apparatus 60a refers to such images that are kept in association with the Device ID and the transport ID provided in the notification. The management apparatus 10a brings the Device ID and the transport ID of the referenced images into association with the emergency ID obtained from the RIS 40a (4a. Bring Patient into Association).

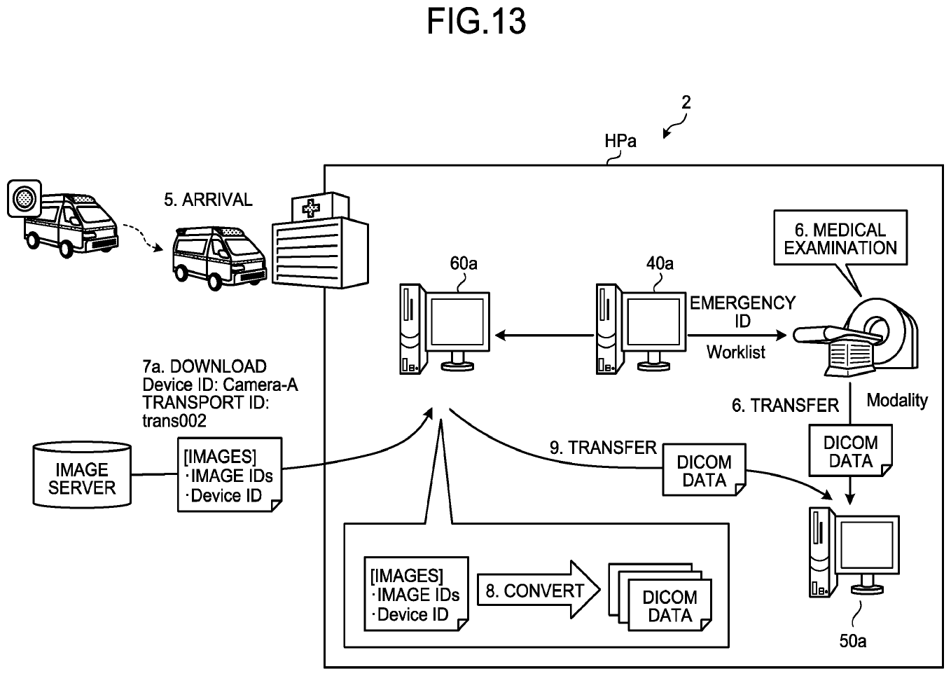

Next, a process performed after the patient arrives at the hospital HPa in the second embodiment will be explained. FIG. 13 is a drawing for explaining the process performed after the arrival of the patient according to the second embodiment. When the patient transported by the ambulance in which the onboard device 70a is mounted arrives at the hospital HPa, the management apparatus 60a downloads the pre-arrival medical information including the images of the patient from the image server 80 (7a. Download). In this situation, the management apparatus 10a downloads images kept in association with the Device ID and the transport ID provided in the notification in advance. The processes thereafter are the same as those illustrated in FIG. 3.

Next, a function of a medical information management system 2 that performs the processes illustrated in FIGS. 12 and 13 will be explained. The management apparatus 60a included in the medical information management system 2 includes the communication interface (I/F) 11, the input I/F 12, the display 13, a storage 64, and a processing circuit 65. In this situation, because a management apparatus 60b connected to the intra-hospital network NWb at the hospital HPb also has the same configuration as that of the management apparatus 60a, the management apparatuses 60a and 60b may each be referred to as a management apparatus 60 hereinafter, without being distinguished from each other.

The storage 64 is connected to the processing circuit 65 and is configured to store therein various types of data. In the present embodiment, the storage 64 stores therein a patient table 641, for example.

Similarly to the patient table 141, the patient table 641 stores therein information keeping the pre-arrival medical information obtained from the image server 80 in correspondence with the emergency ID obtained from the RIS 40. The information stored in the patient table 641 is stored by, for example, a first obtaining function 651, the second obtaining function 152, the converting function 153, and a registering function 654 (explained later).

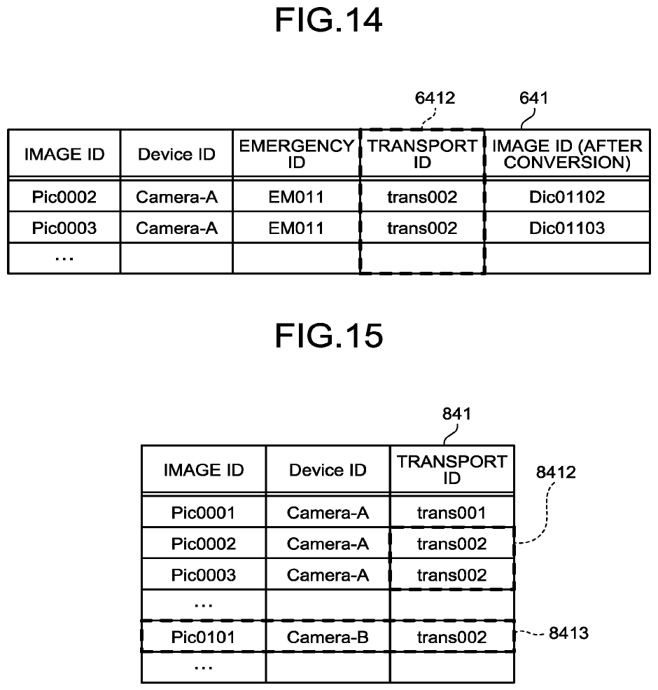

FIG. 14 is a drawing illustrating an example of the patient table according to the second embodiment. For example, as illustrated in FIG. 14, the patient table 641 is information keeping “image IDs”, “Device IDs”, “emergency IDs”, “transport IDs”, and “image IDs (after conversion)” in correspondence with one another. The patient table 641 is different from the patient table 141 according to the first embodiment illustrated in FIG. 5 for storing therein the “transport IDs” instead of the “arrival times”. The patient table 641 illustrated in FIG. 14 stores therein a “transport ID” 6412 issued by the onboard device 70a, for example.

Returning to the description of FIG. 4, the processing circuit 65 is configured to control operations of the entirety of the management apparatus 60 by executing the first obtaining function 651, the second obtaining function 152, the converting function 153, and the registering function 654.

The first obtaining function 651 included in the processing circuit 65 is configured to obtain the pre-arrival medical information from the image server 80. For example, by using the input of the notification about the Device ID and the transport ID or the like as a trigger of the start, the first obtaining function 651 is configured to access the image server 80 via the network NW. The first obtaining function 651 is configured to refer to the images stored in the image server 80 and kept in association with the Device ID and the transport ID. Further, the first obtaining function 151 is configured to output the image IDs of the obtained images and the transport ID to the registering function 654.

Further, by using the input of the notification about the arrival of the patient or the like as a trigger of the start, the first obtaining function 651 is configured to download the image data from the image server 80. The first obtaining function 651 is configured to download the pre-arrival medical information including the data of the images corresponding to the image IDs, the Device ID, and the transport ID that have already been registered in the patient table 641 and to further output the downloaded pre-arrival medical information to the converting function 153.

The registering function 654 is configured to bring the pre-arrival medical information of the transported patient into correspondence with the emergency ID. When having received the image IDs and the transport ID that is output from the first obtaining function 651, the registering function 654 stores the image IDs of the pre-arrival medical information having a matching Device ID and a matching transport ID, into the patient table 641 so as to be kept in association with the emergency ID output from the second obtaining function 152.

Further, as illustrated in FIG. 4, the onboard device 70 includes the camera 21, a transmission data generating unit 72, and the communication I/F 23. Because the onboard devices 70a and 70b have the same configuration as each other, the onboard devices 70a and 70b may each be referred to as an onboard device 70 hereinafter, without being distinguished from each other.

The transmission data generating unit 72 included in the onboard device 70 is configured to generate the pre-arrival medical information to be transmitted to the image server 30, by assigning the identification information to the data of the images and issuing the transport ID. The transmission data generating unit 72 is configured to issue the image IDs for the data of the images, to assign the issued image IDs to the data of the images together with the Device ID assigned to the onboard device 20 and the transport ID, and to transmit the result to the image server 80 via the communication I/F 23. Further, the transmission data generating unit 72 is configured to transmit the transport schedule of the patient including the Device ID and the transport ID to the hospital HPa via the communication I/F 23.

As illustrated in FIG. 4, the image server 80 includes the communication I/F 31, the input I/F 32, the display 33, a storage 84, and a processing circuit 85.

The storage 84 is connected to the processing circuit 85 and is configured to store therein various types of data. The storage 84 stores therein an image table 841, for example.

The image table 841 stores therein information related to the pre-arrival medical information obtained from the onboard device 70. In the present embodiment, the image table 841 is set by an obtaining function 851 and a registering function 852 (explained later), for example.

FIG. 15 is a drawing illustrating an example of the image table according to the second embodiment. As illustrated in FIG. 15, the image table 841 stores therein “image IDs”, “Device IDs”, and “transport IDs” so as to be kept in correspondence with one another, for example. The image table 841 is different from the image table 341 according to the first embodiment illustrated in FIG. 6, for storing therein the “transport IDs” instead of the “arrival times”.

In the image table 841 illustrated in FIG. 15, for example, a “transport ID” 8412 is kept in correspondence with the Device ID “Camera-A” of the onboard device 70a, whereas a “transport ID” 8413 is kept in correspondence with the Device ID “Camera-A” of the onboard device 70b. In other words, the image table 841 illustrated in FIG. 15 stores therein information indicating that the “transport ID” 8412 issued by the onboard device 70a and the “transport ID” 8413 issued by the onboard device 70b are both “trans002”. In this manner, in the present embodiment, the same transport ID may be issued by mutually-different onboard devices 70, in some situations.

Returning to the description of FIG. 4, the processing circuit 85 is configured to control operations of the entirety of the image server 80, by executing the obtaining function 851 and the registering function 852.

The obtaining function 851 included in the processing circuit 85 is configured to obtain the pre-arrival medical information from the onboard device 70. For example, when having received the pre-arrival medical information including the data of the images, from the onboard device 70 via the network NW, the obtaining function 851 is configured to store the data of the images into the storage 84 and is also configured to output the information such as the image IDs, the Device ID, and the transport ID kept in association with the pre-arrival medical information, to the registering function 852.

The registering function 852 is configured to register the information related to the pre-arrival medical information into the image table 841. The registering function 852 is configured to register the image IDs, the Device ID, and the transport ID output from the obtaining function 851 into the image table 841.

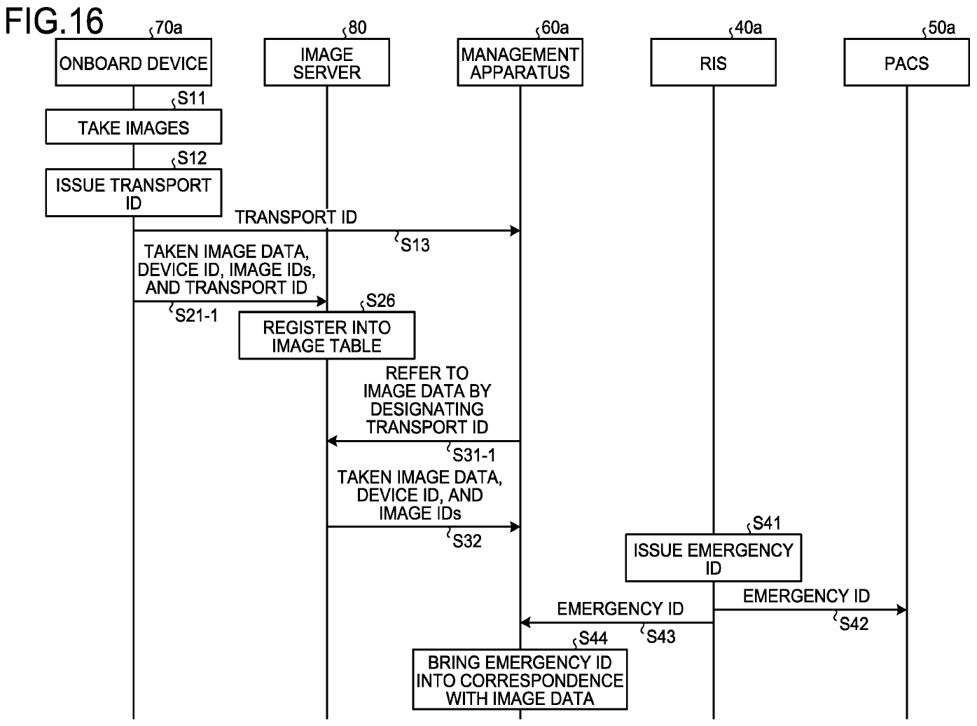

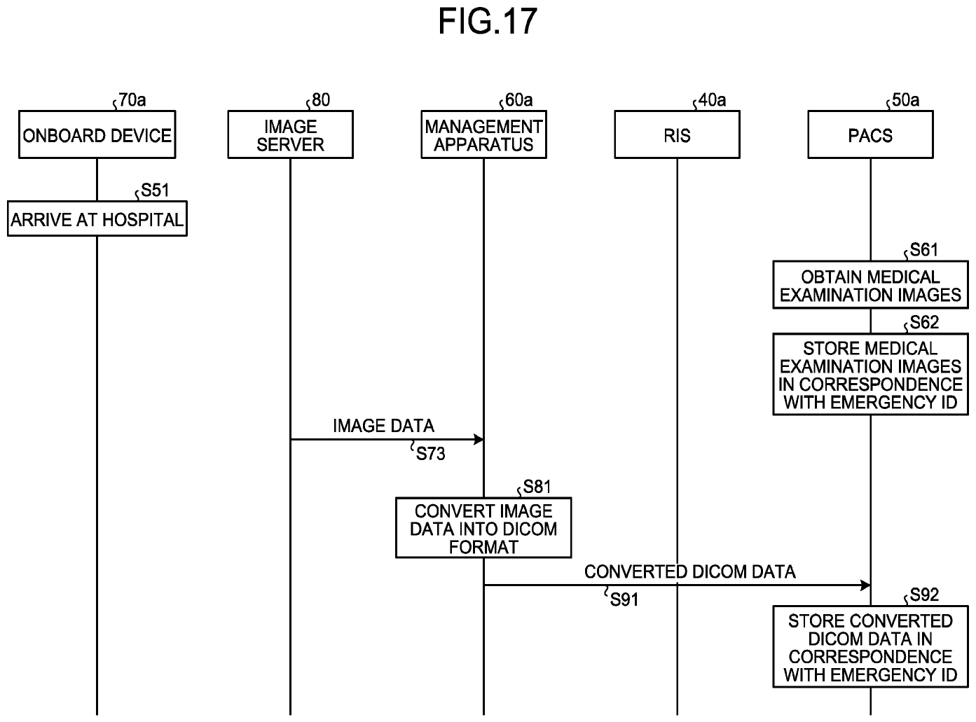

Next, a processing procedure performed by the medical information management system 1 according to the first embodiment will be explained, with reference to FIGS. 16 and 17. FIG. 16 is a sequence chart illustrating an example of the process performed before the arrival of the patient according to the second embodiment. For example, as illustrated in FIG. 16, in the medical information management system 2 according to the present embodiment, the onboard device 70a at first takes images of the patient during the transport (step S11) and issues a transport ID (step S12). The onboard device 70a notifies the management apparatus 60a at the hospital HPa of the issued transport ID (step S13). Subsequently, the onboard device 70a transmits the taken image data to the image server 80, so as to be kept in association with the image IDs, the Device ID, and the transport ID (step S21-1).

Subsequently, when having obtained the image data, the image IDs, the Device ID, and the transport ID, the image server 80 registers the image IDs, the Device ID, and the transport ID into the image table 341 (step S26). After that, when having obtained the Device ID, the processing circuit 65 refers to the images stored in the image server 80 by designating the Device ID and the transport ID (step S31-1). The processing circuit 65 obtains the data of the images corresponding to the Device ID and the transport ID, as well as the Device ID and the image IDs (step S32). The processes thereafter are the same as those illustrated in FIG. 10.

Next, a process performed in the medical information management system 1 after the arrival of the patient will be explained. FIG. 17 is a sequence chart illustrating an example of the process performed after the arrival of the patient according to the second embodiment. When the patient arrives at the hospital (step S51), the processing circuit 15 accesses the image server 80 without receiving a notification of the arrival time and downloads the image data newly registered with the arrival time, together with the arrival time (step S73). The processes thereafter the same as those illustrated in FIG. 11.

As explained above, according to the second embodiment, the pre-arrival medical information includes the images taken of the patient. According to the second embodiment, the first obtaining function 651 is configured to further obtain, as the information kept in association with the pre-arrival medical information, the transport identification information serving as the information identifying the patient transport procedure to the hospital and the device identification information identifying the device that took the images of the patient. The registering function 654 according to the second embodiment is configured to bring the pre-arrival medical information specified by the transport identification information and the device identification information, into association with the patient identification information. Accordingly, also in the medical information management system 2 according to the second embodiment, it is possible to easily bring the data related to the patient acquired during the transport to the hospital into association, at the hospital being the transport destination.

In the second embodiment, the patient being transported is specified by using the set made up of the transport ID and the Device ID. Accordingly, even when the transport ID issued by the onboard device 70a is a duplicate of the transport ID that was already issued by the other different onboard device (i.e., 70b), it is possible to uniquely identify the patient being transported. Consequently, according to the second embodiment, it is possible to enable each of the onboard devices 70 to assign a unique transport ID, without the need to integrally control the assigning of the transport IDs among the plurality of onboard devices 70.

Further, in the second embodiment, another arrangement is also acceptable in which the storage device (the image server 80) stores therein the issued identification information (the transport ID) issued by the extra-order device (the onboard device 70) and uniquely identifying the patient who used the extra-order device, so as to be kept in correspondence with the medical information based on the extra-order device. The processing circuit 15 included in the medical information management apparatus may be configured to obtain the issued identification information and to obtain the medical information kept in correspondence with the issued identification information, as the medical information of the patient who is to use the hospital.

### Third Embodiment

In the second embodiment above, the configuration was explained in which the transported patient is specified by using the set made up of the Device ID of the onboard device 70a and the transport ID uniquely identifying the patient transport procedure; however, the extra-order device that obtains the medical information is not limited to the onboard devices. For example, another configuration is also acceptable in which the medical information obtained by the onboard device 70a is brought into association with medical information obtained by an external extra-order device such as a smartphone or a drone. In a third embodiment, an example will be explained in which, before an onboard device Ca (explained later) obtains images of the patient, pre-arrival medical information obtained by an external extra-order device is further brought into association with the post-arrival medical information.

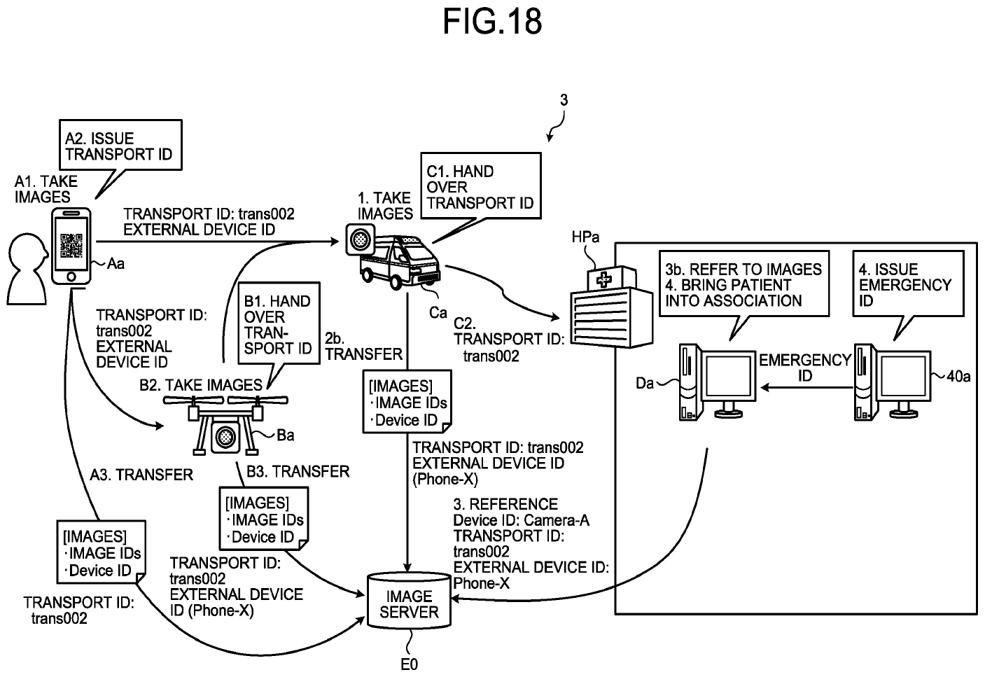

First, in the third embodiment, a process performed before the patient arrives at the hospital HPa illustrated in FIG. 1 will be explained. FIG. 18 is a drawing for explaining the process performed before the arrival of the patient according to the third embodiment. In the embodiments hereinafter, some of the elements that are the same as those illustrated in the drawings explained earlier will be referred to by using the same reference characters, and the duplicate explanations thereof will be omitted. Further, in the following sections, when a smartphone Aa and a drone Ba, which are external extra-order devices, are referred to without being distinguished from each other, the smartphone Aa and the drone Ba may each simply be referred to as an “external device”. Further, in the following sections, when being referred to without being distinguished from each other, the onboard devices and the external devices may each simply be referred to as a “device”. Further, when being distinguished from the Device ID of an onboard device 70, the Device ID of an external device may be referred to as an “external Device ID”. In the present embodiment, an external Device ID “Phone-X” is assigned to the smartphone Aa, whereas an external Device ID “Drone-Y” is assigned to the drone Ba. Each of the external devices is an example of another extra-order device.

As illustrated in FIG. 18, the smartphone Aa takes one or more images of the patient before the ambulance in which the onboard device Ca is mounted arrives, assigns “image IDs” thereto (A1. Take Images), and also issues a transport ID “trans002” (A2. Issue Transport ID). After that, the smartphone Aa transmits the taken images to an image server E0, together with the “image IDs”, the “external Device ID”, and the “transport ID” (A3. Transfer).

Subsequently, before the ambulance in which the onboard device Ca is mounted arrives, the drone Ba arrives at the patient's location and obtains the transport ID issued by the smartphone Aa and the external Device ID (B1. Hand Over Transport ID). The drone Ba takes one or more images of the patient and assigns “image IDs” thereto (B2. Take Images) and transmits the taken images to the image server E0, together with the “image IDs”, the external Device ID of the drone Ba, the “external Device ID of the smartphone Aa”, and the “transport ID: trans002” (B3. Transfer).

After that, when the ambulance in which the onboard device Ca is mounted arrives at the patient's location, the onboard device Ca obtains the transport ID issued by the smartphone Aa and the external Device ID (C1. Hand Over Transport ID). Further, the onboard device Ca takes one or more images of the patient and assigns “image IDs” thereto (1. Take Images) and transmits the obtained transport ID “trans002” to the hospital HPa (C2. Transport ID: trans002). After that, the onboard device Ca transmits the taken images to the image server E0, together with the “image IDs”, the “Device ID of the onboard device Ca”, “the external Device ID of the smartphone Aa”, and the “transport ID: trans002” (A3. Transfer).

Further, the hospital HPa is notified of a schedule of the transport of the patient in the ambulance, together with the Device ID of the onboard device Ca mounted in the ambulance, the external Device ID of the smartphone Aa, and the transport ID. In this situation, a management apparatus Da at the hospital HPa refers to images stored in the image server E0 (3b. Refer to Images). At that time, the management apparatus Da refers to such images that are kept in association with either the Device ID or the external Device ID and the transport ID provided in the notification. The processes thereafter are the same as those illustrated in FIGS. 12 and 13.

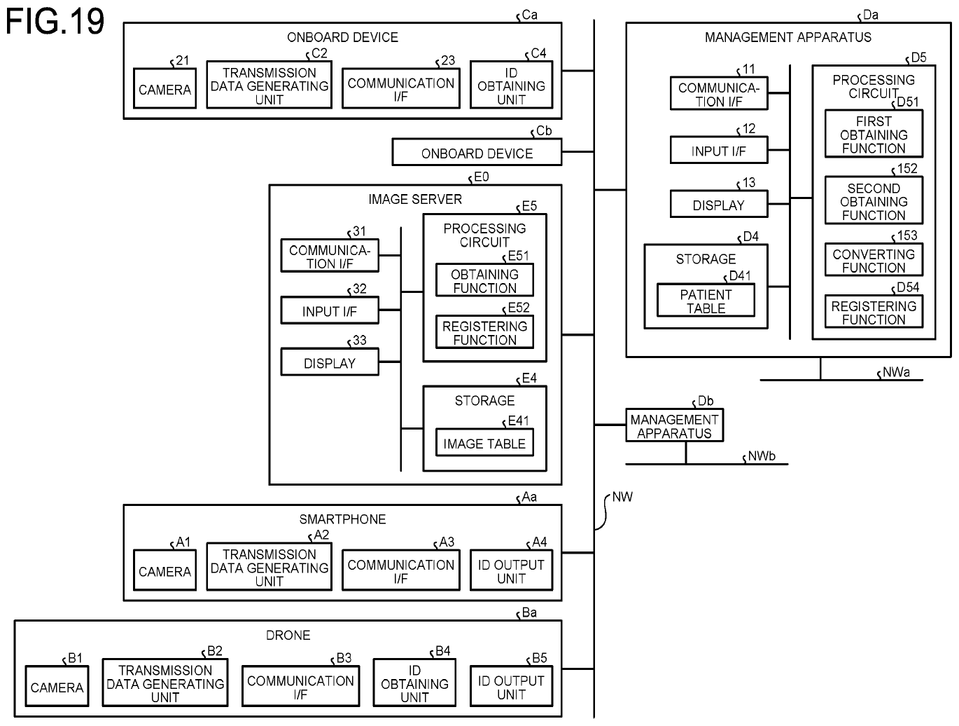

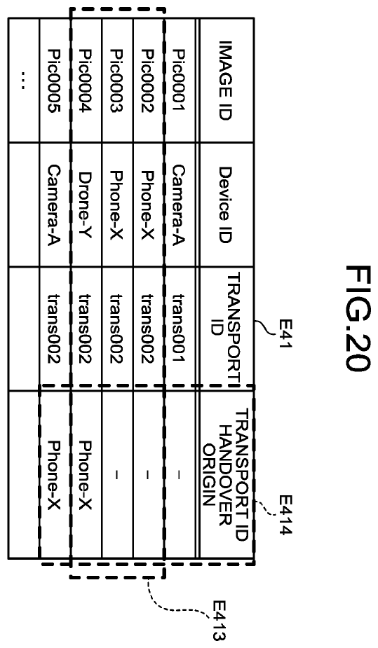

Next, a function of a medical information management system 3 that performs the processes illustrated in FIG. 18 will be explained. FIG. 19 is a block diagram of the medical information management system according to the third embodiment. As illustrated in FIG. 19, the medical information management system 3 according to the present embodiment includes the smartphone Aa, the drone Ba, management apparatuses Da and Db, onboard devices Ca and Cb, and the image server E0. Further, the management apparatus Da is communicably connected to the RIS 40a (not illustrated) and to the PACS 50a (not illustrated), via the intra-hospital network NWa. Similarly, the management apparatus Db is communicably connected to the RIS 40b (not illustrated) and to the PACS 50b (not illustrated), via the intra-hospital network NWb. With reference to FIG. 19, the example was explained in which the one smartphone Aa and the one drone Ba are provided; however, possible embodiments are not limited to this example. It is also acceptable to provide two or more smartphones and/or two or more drones.

Before the ambulance in which the onboard device Ca is mounted arrives, the smartphone Aa is configured to take one or more images of the patient and to transmit pre-arrival medical information including the images and the like, to the image server E0. As illustrated in FIG. 19, the smartphone Aa includes a camera A1, a transmission data generating unit A2, a communication I/F A3, and an ID output unit A4. In this situation, the smartphone Aa is realized by installing an application that has functions corresponding to the transmission data generating unit A2 and the ID output unit A4, in an existing smartphone having a camera function and a communication function, for example.

The camera A1 of the smartphone Aa is configured, similarly to the camera 21 of the onboard device 20, to obtain images of the patient and to output data of the images to the transmission data generating unit A2.

The transmission data generating unit A2 is configured, similarly to the transmission data generating unit 22 of the onboard device 20, to generate the pre-arrival medical information to be transmitted to the image server E0, by assigning the identification information to the data of the images and issuing the transport ID. The transmission data generating unit E2 is configured to issue the image IDs for the data of the images, to assign the issued image IDs to the data of the images, together with the Device ID and the transport ID assigned to the smartphone Aa, and to transmit the result to the image server E0 via the communication I/F A3. Further, the transmission data generating unit A2 is configured to transmit the transport schedule of the patient including the Device ID and the transport ID, to the hospital HPa via the communication I/F A3.

The communication I/F A3 is configured to control transmission and communication of various types of data, to/from and with other apparatuses and devices or the like connected via the network NW, and the like. For example, the communication I/F A3 is configured to transmit the pre-arrival medical information output from the transmission data generating unit 22 to the image server E0. For example, the communication I/F A3 may be realized by using a network card, a network adaptor, or the like. Further, as a standard of the communication, the communication I/F A3 may adopt a standard of near-field wireless communication such as Wi-Fi or Bluetooth (a registered trademark).

The ID output unit A4 is configured to output the external Device ID and the issued transport ID to the drone Ba, the onboard device Ca, and the like. The ID output unit A4 may cause a display (not illustrated) to display a two-dimensional code (e.g., a QR code (a registered trademark)) including information related to the external Device ID and the transport ID or may output the information related to the external Device ID and the transport ID via the communication I/F A3.

Further, before the ambulance in which the onboard device Ca is mounted arrives, the drone Ba is configured to arrive at the site, to take one or more images of the patient, and to transmit pre-arrival medical information including the images, to the image server E0. As illustrated in FIG. 19, the drone Ba includes a camera B1, a transmission data generating unit B2, a communication I/F B3, an ID obtaining unit B4, and an ID output unit B5. The drone Ba is realized by installing an application that has functions corresponding to the transmission data generating unit B2, the ID obtaining unit B4, and the ID output unit B5, in an existing drone having a camera function and a communication function, for example.

The camera B1 of the drone Ba is configured, similarly to the camera A1 of the smartphone Aa, to obtain the images of the patient and to output data of the images to the transmission data generating unit B2.

The transmission data generating unit B2 is configured, similarly to the transmission data generating unit A2 of the smartphone Aa, to generate the pre-arrival medical information to be transmitted to the image server E0 by assigning the identification information to the data of the images and issuing the transport ID. The transmission data generating unit E2 is configured to issue the image IDs to the data of the images, to assign the issued image IDs to the data of the images together with the Device ID of the drone Ba and the transport ID, and to transmit the result to the image server E0 via the communication I/F B3. Further, the transmission data generating unit B2 is configured to transmit the transport schedule of the patient including the Device ID and the transport ID, to the hospital HPa, via the communication I/F B3.

When the ID obtaining unit B4 has obtained the external Device ID and the transport ID from the smartphone Aa, for example, the transmission data generating unit B2 is configured to assign the external Device ID of the smartphone Aa and the obtained transport ID to the data of the images together with the image IDs and the Device ID of the drone Ba and to further transmit the result to the image server E0 via the communication I/F B3. Further, the transmission data generating unit B2 is configured to transmit the Device ID, the external Device ID of the smartphone Aa, and the transport schedule of the patient including and the transport ID received from the smartphone Aa, to the hospital HPa via the communication I/F B3.

The communication I/F B3 is configured to control transmission and communication of various types of data, to/from and with other apparatuses and devices or the like connected via the network NW. For example, the communication I/F B3 is configured to transmit the pre-arrival medical information output from the transmission data generating unit 22, to the image server E0. For example, the communication I/F B3 may be realized by using a network card, a network adaptor, or the like. Further, as a standard of the communication, the communication I/F B3 may adopt a standard of near-field wireless communication such as Wi-Fi or Bluetooth (a registered trademark).

The ID obtaining unit B4 is configured to obtain an external Device ID and a transport ID from other external devices such as the smartphone Aa. The ID obtaining unit B4 is configured to obtain the external Device ID and the transport ID by, for example, taking an image of a two-dimensional code displayed on the display of the smartphone Aa or receiving information transmitted from the smartphone Aa via the communication I/F B3.

The ID output unit B5 is configured, similarly to the ID output unit A4 of the smartphone Aa, to output the Device ID and the issued transport ID to the onboard device Ca or the like. For example, when the ID obtaining unit B4 has obtained the external Device ID and the transport ID from the smartphone Aa, the ID output unit B5 may output information related to the external Device ID of the smartphone Aa and the obtained transport ID.

Further, the onboard device Ca includes, as illustrated in FIG. 19, the camera 21, a transmission data generating unit C2, the communication I/F 23, and an ID obtaining unit C4. Because the onboard devices Ca and Cb have the same configuration as each other, the onboard devices Ca and Cb may each be referred to as an onboard device C hereinafter, without being distinguished from each other.

The transmission data generating unit C2 is configured, similarly to the transmission data generating unit 22, to generate the pre-arrival medical information to be transmitted to the image server 30, by assigning identification information to the data of the images. For example, when the ID obtaining unit C4 has obtained an external Device ID and a transport ID from either the smartphone Aa or the drone Ba, the transmission data generating unit C2 may assign the external Device ID and the obtained transport ID to the data of the images and transmit the result to the image server E0 via the communication I/F 23. Further, the transmission data generating unit C2 may transmit the transport schedule of the patient including the external Device ID and the obtained transport ID, to the hospital HPa via the communication I/F 23.

The ID obtaining unit C4 is configured to obtain am external Device ID and a transport ID from an external device such as the smartphone Aa, the drone Ba, or the like. The ID obtaining unit C4 is configured to obtain the external Device ID and the transport ID by, for example, taking an image of a two-dimensional code displayed on a display of the external device or receiving information transmitted from the external device via the communication I/F B3.

The management apparatus Da includes the communication I/F 11, the input I/F 12, the display 13, a storage D4, and a processing circuit D5. In this situation, because the management apparatus Db connected to the intra-hospital network NWb at the hospital HPb also has the same configuration as that of the management apparatus Da, the management apparatuses Da and Db may each be referred to as a management apparatus D hereinafter, without being distinguished from each other.

The storage D4 is connected to the processing circuit D5 and is configured to store therein various types of data. In the present embodiment, for example, the storage D4 stores therein a patient table D41. The patient table D41 is, similarly to the patient table 641, information keeping “image IDs”, “Device IDs”, “emergency IDs”, “transport IDs”, and “image IDs (after conversion)” in correspondence with one another. As a Device ID kept in association with the pre-arrival medical information obtained by an external device, for example, the patient table D41 may, in some situations, store therein an external Device ID, instead of the Device ID of the onboard device Ca.

The processing circuit D5 is configured to control operations of the entirety of the management apparatus D, by executing a first obtaining function D51, the second obtaining function 152, the converting function 153, and a registering function D54.

The first obtaining function D51 included in the processing circuit D5 is configured to obtain the pre-arrival medical information from the image server E0. For example, by using the input of the notification about the Device ID, the external Device ID, and the transport ID or the like as a trigger of the start, the first obtaining function D51 is configured to access the image server E0 via the network NW. The first obtaining function D51 is configured to refer to the images stored in the image server E0 and kept in association with either the Device ID or the external Device ID and the transport ID. Further, the first obtaining function 151 is configured to output the image IDs of the obtained images and the transport ID to the registering function D54.

Further, the first obtaining function D51 is configured to download image data from the image server D0, by using the input of the notification about the arrival of the patient or the like as a trigger of the start. The first obtaining function D51 is configured to download the pre-arrival medical information including the data of the images corresponding to the image IDs, either the Device ID or the external Device ID, and the transport ID that have already been registered in the patient table D41 and to further output the downloaded pre-arrival medical information to the converting function 153.

The registering function D54 is configured to bring the pre-arrival medical information of the transported patient into association with the emergency ID. When having received the image IDs and the transport ID that is output from the first obtaining function D51, the registering function D54 is configured to store the images IDs of the pre-arrival medical information having either a matching Device ID or a matching external Device ID and a matching transport ID into the patient table D41, so as to be kept in association with the emergency ID output from the second obtaining function 152.

As illustrated in FIG. 4, the image server E0 includes the communication I/F 31, the input I/F 32, the display 33, a storage E4, and a processing circuit E5.

The storage E4 is connected to the processing circuit E5 and is configured to store therein various types of data. For example, the storage E4 stores therein an image table E41.

The image table E41 stores therein information related to the pre-arrival medical information obtained from the onboard device 70. In the present embodiment, the image table E41 is set, for example, by an obtaining function E51 and a registering function E52 (explained later).

FIG. 20 is a drawing illustrating an example of the image table according to the third embodiment. As illustrated in FIG. 20, for example, the image table E41 further stores therein “transport ID handover origins” in addition to the “image IDs”, the “Device IDs”, and the “transport IDs” so as to be kept in correspondence with one another. A “transport ID handover origin” E414 indicates the external Device ID of an external device that issued a transport ID, in the situation where either an onboard device or an external device indicated under “Device IDs” received the transport ID handed over by the other external device, instead of issuing the transport ID by itself.

In the image table E41 illustrated in FIG. 20, the Device IDs registered in the three records E414 corresponding to the image IDs “Pic0002”, “Pic0003”, and “Pic0004” are “Phone-X” and “Drone-Y”, and not “Camera-A”. In other words, the three records E414 indicate that the corresponding pre-arrival medical information was obtained by either the smartphone Aa or the drone Ba, each being an external device, and not by the onboard device Ca.

In the image table E41 illustrated in FIG. 20, for example, the record of the image ID “Pic0004” is a record related to an image obtained by the drone Ba identified with the Device ID “Drone-Y” stores therein information indicating that the transport ID was issued by the smartphone Aa identified with the Device ID “Phone-X”. Similarly, for example, the record of the image ID “Pic0005” is a record related to an image obtained by the onboard device Ca identified with the Device ID “Camera-A” and stores therein information indicating that the transport ID was issued by the smartphone Aa identified with the Device ID “Phone-X”.

Returning to the description of FIG. 4, the processing circuit E5 is configured to control operations of the entirety of the image server E0, by executing an obtaining function E51 and a registering function E52.

The obtaining function E51 included in the processing circuit E5 is configured to obtain the pre-arrival medical information from the onboard device 70. For example, when having received the pre-arrival medical information including the data of the images from the smartphone Aa, the drone Ba, or the onboard device Ca via the network NW, the obtaining function E51 is configured to store the data of the images into the storage E4 and to also output information such as the image IDs, Device ID, the external Device ID, and the transport ID kept in association with the pre-arrival medical information, to the registering function E52.

The registering function E52 is configured to register the information related to the pre-arrival medical information to the image table E41. For example, the registering function E52 is configured to register the image IDs, the Device ID, the external Device ID, and the transport ID output from the obtaining function E51 into the image table E41. When the obtaining function E51 further outputs the external Device ID in addition to the Device ID, the registering function E52 stores the external ID into the image table E41, as a “transport ID handover origin”.

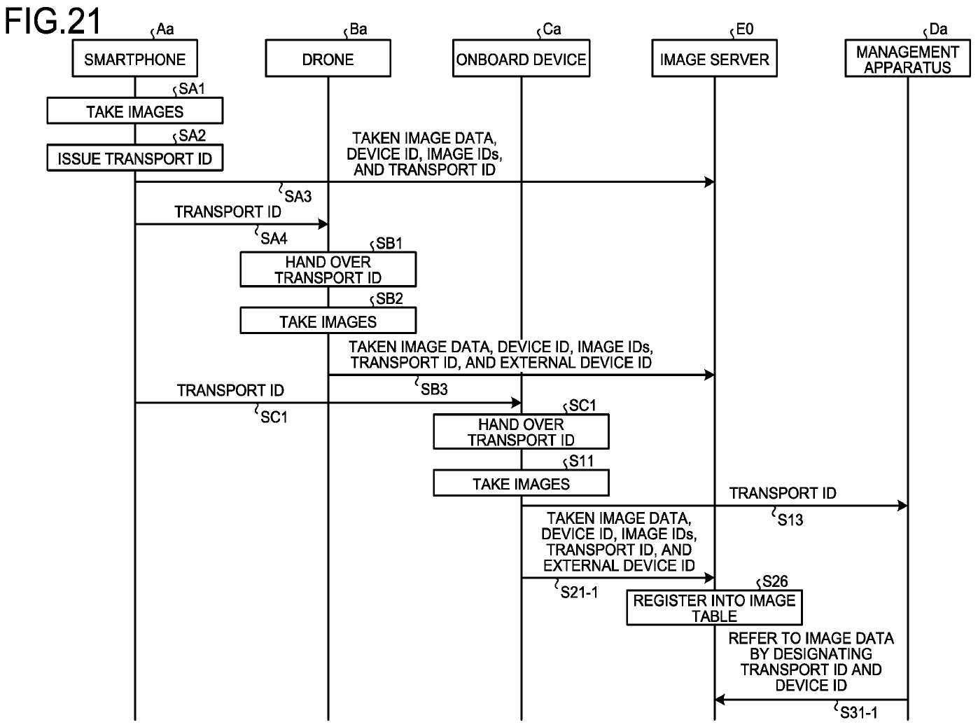

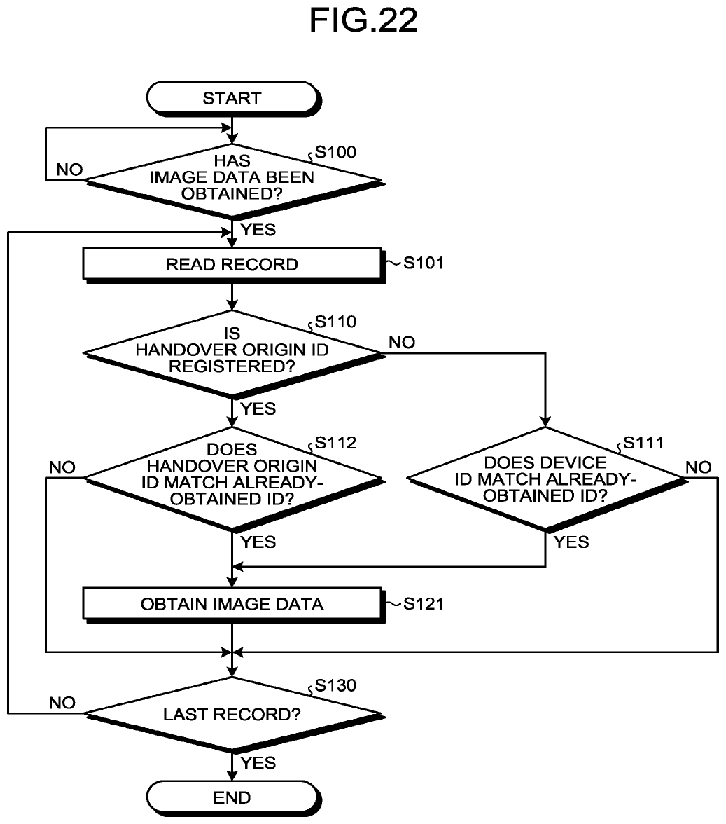

Next, a processing procedure performed by the medical information management system 3 according to the third embodiment will be explained, with reference to FIGS. 21 and 22. FIG. 21 is a sequence chart illustrating an example of a process performed before the arrival of the patient according to the third embodiment. For example, as illustrated in FIG. 21, in the medical information management system 2 according to the present embodiment, the smartphone Aa at first takes images of the patient prior to the arrival of the onboard device Ca (step SA1) and issues a transport ID (step SA2). The smartphone Aa transmits the taken image data to the image server E0 so as to be kept into association with image IDs, the Device ID, and the transport ID (step SA3).

Subsequently, upon arrival at the patient's location prior to the arrival of the onboard device Ca, the drone Ba obtains the external Device ID and the transport ID from the smartphone Aa (step SA4) so as to receive the transport ID handed over (step SB1). After that, the drone Ba takes images of the patient (step SB2) and transmits the taken image data to the image server E0 so as to be kept in association with image IDs, the Device ID, and the external Device ID and the transport ID obtained from the smartphone Aa (step SB3).

After that, upon arrival at the patient's location, the onboard device Ca obtains the external Device ID and the transport ID from the smartphone Aa and receives the transport ID handed over (step SC1). Further, the onboard device Ca takes images of the patient (step S11), transmits the taken image data to the image server E0 so as to be kept into association with the image IDs, the Device ID, and the transport ID and the external ID obtained from the smartphone Aa (step S21-1). The processes thereafter are the same as those illustrated in FIGS. 16 and 17.

Next, a process performed by the management apparatus Da at step S73 illustrated in FIG. 17 to obtain the image data from the image server E0 will be explained. FIG. 22 is a flowchart illustrating an example of the image data obtaining process according to the third embodiment. In this situation, steps S100 through S130 in FIG. 22 are realized, for example, as a result of the processing circuit D5 of the management apparatus Da reads and executes the program corresponding to the registering function D54 from the storage D4.

At first, the processing circuit D5 stands by until an image data obtaining instruction is received (step S100: No). When having received an image data obtaining instruction (step S100: Yes), the processing circuit D5 accesses the image server E0 and reads such a record that has a matching transport ID, from the image table E41 (step S101).

Subsequently, the processing circuit D5 judges whether or not a handover origin ID is registered in the read record (step S110). When having determined that the read record does not have a handover origin ID registered therein (step S110: No), the processing circuit D5 judges whether or not the Device ID registered in the read record matches an already-obtained Device ID (step S111). When having determined that the Device ID registered in the read record matches an already-obtained Device ID (step S111: Yes), the processing circuit D5 downloads the image data corresponding to the record (step S121). After that, the procedure proceeds to step S130. On the contrary, when having determined that the Device ID registered in the read record matches no already-obtained Device ID (step S111: No), the processing circuit D5 proceeds to step S130.

Returning to the description of step S110, when having determined that the read record has a handover origin ID registered therein (step S110: Yes), the processing circuit D5 judges whether or not the handover origin ID matches an already-obtained external Device ID (step S112).

When having determined that the handover origin ID matches an already-obtained external Device ID (step S112: Yes), the processing circuit D5 downloads the image data corresponding to the record (step S121). After that, the procedure proceeds to step S130. On the contrary, when having determined that the handover origin ID matches no already-obtained external Device ID (step S112: No), the processing circuit D5 proceeds to step S130.

Subsequently, the processing circuit D5 judges whether or not the read record is the last record in the image table E41 (step S130). When having determined that the read record is not the last record in the image table E41 (step S130: No), the processing circuit D5 returns to step S101 and reads the next record. On the contrary, when having determined that the read record is the last record in the image table E41 (step S130: Yes), the processing circuit D5 ends the process.

As explained above, according to the third embodiment, the pre-arrival medical information further includes the images of the patient taken by the external device, which is a device other than the onboard device kept in association with the transporter transporting the patient to the hospital. According to the third embodiment, the first obtaining function D51 is configured to further obtain, as the information to be kept in association with the pre-arrival medical information, the device identification information of the external device and the transport identification information kept in association with the external device. The registering function D54 according to the third embodiment is configured to bring the pre-arrival medical information specified by the device identification information of the external device and the transport identification information kept in association with the external device, into association with the device identification information of the device kept in association with the transporter and the patient identification information. Consequently, in the medical information management system 3 according to the third embodiment, it is possible to also easily bring the data related to the patient obtained before the arrival of the ambulance into association, at the hospital being the transport destination.

Further, in the third embodiment, another arrangement is acceptable in which the extra-order device (the onboard device C) receives the issued identification information issued and handed over by another extra-order device (an external device). The storage device (the image server E0) may store therein the issued identification information (the transport ID) uniquely identifying a patient who is shared among a plurality of extra-order devices so as to be kept in correspondence with the medical information based on the extra-order devices. The processing circuit 15 of the medical information management apparatus may obtain the issued identification information and may further obtain the medical information kept in correspondence with the issued identification information as medical information of the patient who is to use the hospital.

According to the third embodiment, the patient being transported is specified by using either the set made up of a transport ID and a Device ID or the set made up of a transport ID and an external Device ID. According to the third embodiment, the transport ID and the external Device ID assigned to the pre-arrival medical information by the external device are handed over to the onboard device Ca and are also assigned to the pre-arrival medical information obtained by the onboard device Ca. In other words, even when the transport ID assigned to the pre-arrival medical information by the external device is the same as the transport ID already issued by the onboard device Ca, it is possible to uniquely identify the patient being transported.

In the third embodiment, possible configurations for uniquely identifying the patient being transported are not limited to the example described above. For instance, the image server E0 may replace the transport ID handed over from the external device to the onboard device Ca with a transport ID newly issued by the onboard device Ca. Further, the external device may issue a transport ID that is not a duplicate of any of the transport IDs issued by the onboard device Ca, so that the onboard device Ca conveniently uses the issued transport ID without any further change. Further, the external device may use the external Device ID as a transport ID, instead of newly issuing a transport ID.

Further, the transport ID issued by the external device may be a transport ID that is also unique among other external devices and onboard devices. In that situation, it is possible to uniquely identify the patient being transported, without specifying a Device ID. Accordingly, the management apparatus Da is able to specify the pre-arrival medical information, without the need to cause the onboard device Ca to obtain the external Device ID from the smartphone Aa or the drone Ba.

### Fourth Embodiment

Incidentally, the information included in the medical information does not necessarily have to be images and may be other information such as an electrocardiogram or a vital sign. Similarly, extra-order devices such as the onboard devices and the external devices and the like that acquire the medical information do not necessarily have to be cameras and the like that take images and may instead be a device configured to obtain other types of information such as a device that takes an electrocardiogram or the heart rate of the patient. Further, the example was explained above in which the onboard devices and the transporters are in one-to-one correspondence; however, possible embodiments are not limited to this example. Another configuration is acceptable in which a plurality of onboard devices mounted in one transporter such as an ambulance or a medical emergency helicopter are each kept in correspondence with the transporter. Further, possible configurations used for handing over the information identifying the patient corresponding to the medical information are not limited to the example in the third embodiment where the transport ID and the Device ID are used. In a fourth embodiment, a configuration will be explained in which a plurality of pieces of medical information concerning to a same patient acquired by a same or a different extra-order device is identified, by associating medical information including information other than images without depending on the transport ID and the Device IDs used in the third embodiment.

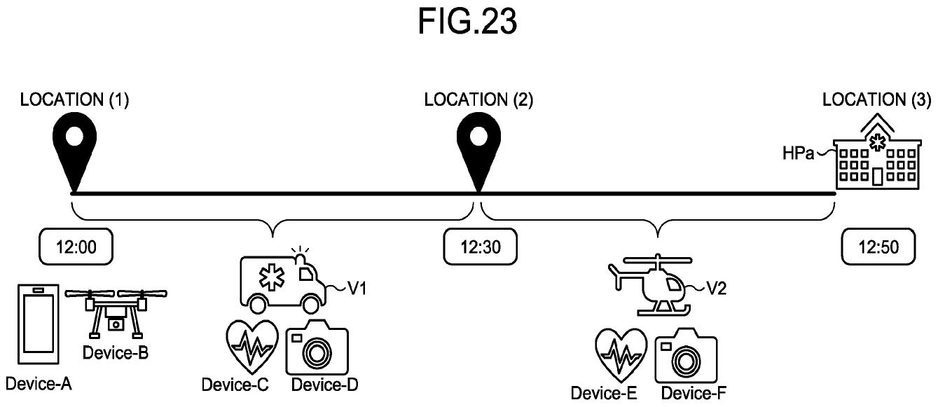

First, in the fourth embodiment, a process performed before a patient arrives at the hospital HPa illustrated in FIG. 1 will be explained. FIG. 23 is a drawing for explaining the process before the arrival of the patient according to the fourth embodiment. In FIG. 23, the patient (not illustrated) is transported from location (1) via location (2) to the hospital HPa in location (3). Until an ambulance V1 arrives at location (1), medical information of the patient is obtained by, for example, a Device-A and a Device-B, which are external devices. The Device-A is a smartphone, for example. The Device-B is a drone, for example.

After that, when the ambulance V1 arrives at location (1) at 12:03, for example, the patient is transported from location (1) to location (2) by the ambulance V1. At that time, medical information of the patient is obtained, for example, by a Device-C and a Device-D, which are onboard devices mounted in the ambulance V1. The Device-C is a heart rate monitor, for example. The Device-D is a camera, for example.

After that, when the ambulance V1 arrives at location (2) from location (1) at 12:30, the patient is transferred onto a medical emergency helicopter V2 and is further transported to the hospital HPa. At that time, medical information of the patient is obtained, for example, by a Device-E and a Device-F, which are onboard devices mounted in the medical emergency helicopter V2. The Device-E is a heart rate monitor, for example. The Device-F is a camera, for example. After that, the patient arrives at the hospital HPa in location (3) at 12:50.

As illustrated in FIG. 23, when the plurality of mutually-different devices obtain pieces of medical information, when the times and the locations at which the pieces of medical information were obtained are close to each other the same, it is possible to conjecture that the pieces of medical information obtained by the mutually-different devices correspond to the same patient. Further, when the time at which a certain device starts up an application (“app”) used for transmitting the medical information is close to the time at which another device shuts down the application, it is also similarly possible to conjecture that pieces of medical information obtained by the mutually-different devices correspond to the same patient. Further, when pieces of biological information (e.g., fingerprints or the iris of the patient) or pieces of identification information assigned to the patient that are included in pieces of medical information obtained by mutually-different devices match each other, it is possible to specify that the pieces of medical information obtained by the mutually-different devices correspond to the same patient.

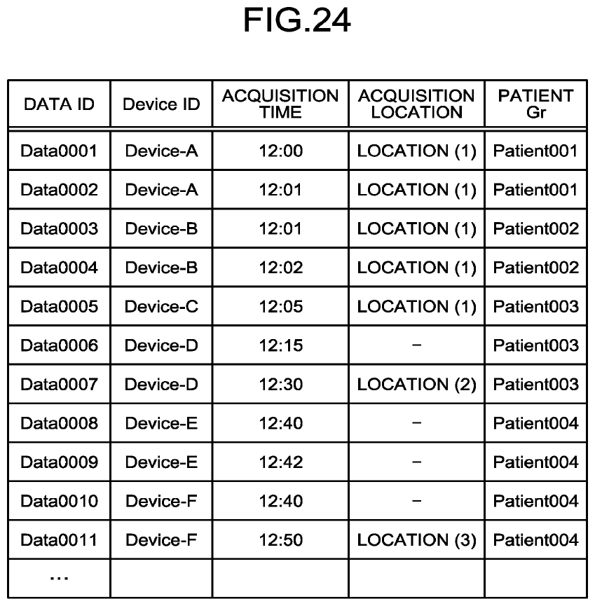

The medical information acquired in the process performed before the arrival of the patient illustrated in FIG. 23 will be explained with reference to FIG. 24. FIG. 24 is a drawing illustrating an example of medical information data according to the fourth embodiment. The medical information data illustrated in FIG. 24 is stored in a medical information table I41 (explained later), for example.

In FIG. 24, “data IDs” are each an identifier assigned to the obtained medical information and uniquely identifying the medical information. “Device IDs” are each an identifier uniquely identifying either an onboard device or an external device that obtained the medical information. Each of the “acquisition times” and the “acquisition locations” indicate the time at which the medical information was obtained and position information of the location at which the medical information was acquired, respectively. The “patient Gr” are each an identifier being temporarily assigned to the medical information and identifying the patient. In this situation, the acquisition times may each include a date, for example. The acquisition locations may be registered in a different format such as an address or longitude and latitude. Further, when it is impossible to obtain the position information corresponding to the time when the medical information was acquired, the item under the “acquisition location” is left blank. In this situation, the “patient Gr” is an example of the issued identification information.

In the example illustrated in FIG. 24, the device identified with the Device ID “Device-A” obtains pieces of medical information of a patient at “12:00” and “12:01” at “location (1)”. Further, the patient Gr “Patient001” is assigned to the two obtained pieces of medical information. Similarly, the device identified with the Device ID “Device-B” obtains pieces of medical information of a patient at “12:01” and “12:02” at “location (1)”. Further, the patient Gr “Patient002” is assigned to the two obtained pieces of medical information. Also, the device identified with the Device ID “Device-C” obtains a piece of medical information of a patient at “12:05” at “location (1)”. Further, the patient Gr “Patient003” is assigned to the obtained piece of medical information. Further, the device identified with the Device ID “Device-D” obtains a piece of medical information of a patient at “12:15” at an unidentified location and obtains another piece of medical information of a patient at “12:30” at “location (2)”. Further, the patient Gr “Patient003” is assigned to the two obtained pieces of medical information. Further, the device identified with the Device ID “Device-E” obtains pieces of medical information of a patient at “12:40” and “12:42” at unidentified locations. Further, the patient Gr “Patient004” is assigned to the two obtained pieces of medical information. Similarly, the device identified with the Device ID “Device-F” obtains a piece of medical information of a patient at “12:40” at an unidentified location and obtains a piece of medical information of a patient at “12:50” at “location (3)”. Further, the patient Gr “Patient004” is assigned to the two obtained pieces of medical information.

As explained above, in the present embodiment, the medical information obtained by the “Device-C” and the medical information obtained by the “Device-D”, both devices being mounted in the same transporter (i.e., the ambulance V1), are assigned to the same patient Gr. Similarly, the medical information obtained by the “Device-E” and the medical information obtained by the “Device-F”, both devices being mounted in the same transporter (i.e., the medical emergency helicopter V2), are assigned to the same patient Gr. In other words, in the present embodiment, the pieces of medical information obtained at times close to each other by the devices kept in correspondence with the same transporter are assigned to the same patient Gr. The reason is that it is possible to conjecture that the pieces of medical information obtained by the same transporter at such points in time that are close to each other were obtained from the same patient.

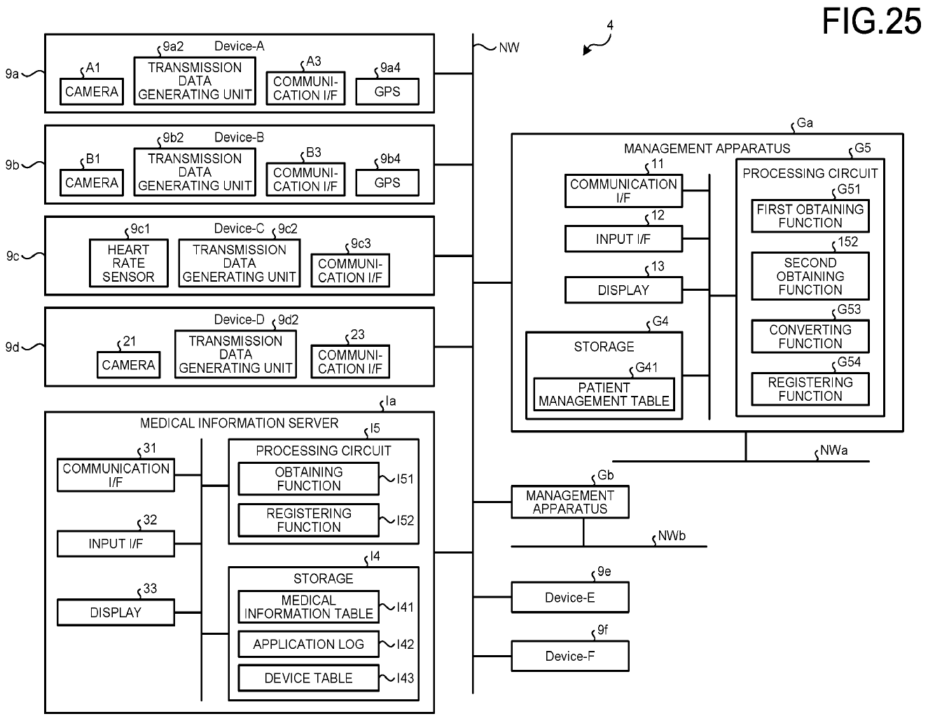

Next, a configuration of a medical information management system configured to process the medical information illustrated in FIG. 24 will be explained, with reference to FIG. 25. FIG. 25 is a block diagram of the medical information management system according to the fourth embodiment. In the embodiments hereinafter, some of the elements that are the same as those illustrated in the drawings explained earlier will be referred to by using the same reference characters, and the duplicate explanations thereof will be omitted.

As illustrated in FIG. 25, a medical information management system 4 according to the present embodiment includes the six devices (Device-A, Device-B, Device-C, Device-D, Device-E and Device-F), two management apparatuses Ga and Gb, and a medical information server Ia. Further, the management apparatus Ga is communicably connected to the RIS 40a (not illustrated) and to the PACS 50a (not illustrated), via the intra-hospital network NWa. Similarly, the management apparatus Gb is communicably connected to the RIS 40b (not illustrated) and to the PACS 50b (not illustrated), via the intra-hospital network NWb. Although FIG. 25 illustrates the example in which the six devices are provided, the number of devices is not limited to this example.

In FIG. 25, the management apparatus Ga connected to the intra-hospital network NWa at the hospital HPa includes the communication I/F 11, the input I/F 12, the display 13, a storage G4, and a processing circuit G5. The storage G4 is connected to the processing circuit G5 and is configured to store therein various types of data. In the present embodiment, the storage G4 stores therein a patient table G41, for example.

The patient table G41 stores therein information keeping the medical information obtained from the medical information server Ia in correspondence with the emergency ID obtained from the RIS 40. In the present embodiment, the information stored in the patient table G41 is stored or updated by, for example, a first obtaining function G51, the second obtaining function 152, a converting function G53, and a registering function G54, for example.

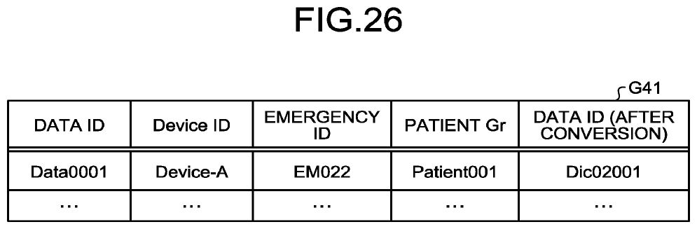

FIG. 26 is a drawing illustrating an example of the patient table according to the fourth embodiment. For example, the patient table G41 is, as illustrated in FIG. 26, information keeping “data IDs”, “Device IDs”, “emergency IDs”, “patient Gr”, and “data IDs (after conversion)” in correspondence with one another. In this situation, the “data IDs” and the “Device IDs” in FIG. 26 are each an identifier uniquely identifying medical information and the device that obtained the medical information, respectively. The “emergency IDs” are each an emergency ID obtained from the RIS 40. The “Patient Gr” are each an identifier being temporarily assigned to the medical information and identifying the patient. The “data IDs (after conversion)” are each an identifier uniquely identifying the medical information converted into the DICOM format. For example, the record illustrated in FIG. 26 indicates that the medical information obtained by the device identified with the Device ID “Device-A” and identified with the data ID “Data0001” and the patient Gr “Patient001” has the emergency ID “EM022” assigned thereto. Further, the record indicates that the medical information was converted into data in the DICOM format, and “Dic02001” was assigned thereto as a data ID after the conversion.

Returning to the description of FIG. 25, the processing circuit G5 is configured to control operations of the entirety of the management apparatus Ga, by executing the first obtaining function G51, the second obtaining function 152, the converting function G53, and the registering function G54.

The first obtaining function G51 included in the processing circuit G5 is configured to obtain the medical information from the medical information server Ia. For example, the first obtaining function G51 is configured to access the medical information server Ia via the network NW, with the input of the notification about the Device ID or the like as a trigger. The first obtaining function G51 is configured to refer to the medical information stored in the medical information server Ia, which is kept in association with the Device ID. Further, the first obtaining function G51 is configured to download the medical information data from the medical information server Ia, with the input of a notification about the arrival of the patient or the like as a trigger. The first obtaining function G51 is configured to download medical information including the data of the medical information corresponding to the medical information ID and the Device ID that have already been registered in the patient table G41 and to further output the downloaded medical information to the converting function G53.

The converting function G53 is configured to convert the medical information data into data in the DICOM format. When having received the medical information that is output from the first obtaining function G51, the converting function G53 is configured to convert the medical information into the data in the DICOM format, together with the data ID, the Device ID, and the emergency ID kept in association therewith. The converting function G53 is configured to transmit the converted medical information to the PACS 50a.

The registering function G54 is configured to bring the medical information of the transported patient into correspondence with the emergency ID. When having received the medical information ID and the patient Gr that is output from the first obtaining function G51, the registering function G54 is configured to store the data IDs, the Device ID, and the patient Cr into the patient table G41 so as to be kept in association with the emergency ID output from the second obtaining function 152.

Further, as illustrated in FIG. 25, a Device-A 9a includes the camera A1, a transmission data generating unit 9a2, the communication I/F A3, and a Global Positioning System (GPS) 9a4. The Device-A 9a is realized by installing an application (“app”) having a function corresponding to the transmission data generating unit 9a2 in an existing smartphone having a camera function, a communication function, and a GPS function, for example.

The transmission data generating unit 9a2 is configured to assign a data ID and a patient Gr to the data of the images obtained by the camera A1, as identification information. Further, the transmission data generating unit 9a2 is configured to obtain position information from the GPS 9a4 and appends the position information to the data of the images. Further, the transmission data generating unit 9a2 is configured to generate the medical information to be transmitted to the medical information server Ia by assigning the Device ID to the data of the images and to transmit the medical information to the medical information server Ia via the communication I/F A3.

Further, when detecting a start-up or a shut-down of the application installed in the Device-A 9a, the transmission data generating unit 9a2 is configured to transmit the time at which the application was started up or shut down and the position information at the point in time, to the medical information server Ia, via the communication I/F A3.

The GPS 9a4 is configured to detect the position information of the Device-A 9a and to output the position information to the transmission data generating unit 9a2.

Further, as illustrated in FIG. 25, a Device-B 9b includes the camera B1, a transmission data generating unit 9b2, the communication I/F B3, and a GPS 9b4. The Device-B 9b is realized by installing an application (“app”) having a function corresponding to the transmission data generating unit 9b2 in an existing drone having a camera function, a communication function, and a GPS function, for example.

The transmission data generating unit 9b2 is configured, similarly to the transmission data generating unit 9a2 of the Device-A 9a, to assign a data ID and a patient Gr to the data of the images obtained by the camera B1, as identification information. Further, the transmission data generating unit 9b2 is configured to obtain position information from the GPS 9b4 and to append the position information to the data of the images. Further, the transmission data generating unit 9b2 is configured to generate medical information to be transmitted to the medical information server Ia by assigning the Device ID to the data of the images and to transmit the medical information to the medical information server Ia via the communication I/F B3.

Further, when detecting a start-up or a shut-down of the application installed in the Device-B 9b, the transmission data generating unit 9b2 is configured to transmit the time at which the application was started up or shut down and the position information at the point in time, to the medical information server Ta, via the communication I/F B3.

The GPS 9b4 is configured to detect the position information of the Device-B and to output the position information to the transmission data generating unit 9b2.

Further, as illustrated in FIG. 25, the Device-C 9c includes a heart rate sensor 9c1, a transmission data generating unit 9c2, and a communication I/F 9c3. The Device-C 9c is, for example, a heart rate sensor mounted in the ambulance V1. The Device-C and the Device-E have the same configuration as each other except for being mounted in mutually-different transporters. Thus, the configuration of the Device-C will be explained below, and detailed explanations of the Device-E will be omitted.

The heart rate sensor 9c1 is a publicly-known heart rate sensor, for example, and is configured to detect a heart rate of the patient, and to output the detected heart rate to the transmission data generating unit 9c2.

The transmission data generating unit 9c2 is configured to assign a data ID and a patient Gr to data of the heart rate obtained from the heart rate sensor 9c1, as identification information. Further, the transmission data generating unit 9c2 is configured to obtain position information from the GPS mounted in the ambulance V1 and to append the position information to the data of the heart rate. Further, the transmission data generating unit 9c2 is configured to generate medical information to be transmitted to the medical information server Ia by assigning the Device ID to the data of the heart rate and to transmit the medical information to the medical information server Ia via the communication I/F 9c3.

The communication I/F 9c3 is configured to control transmission and communication of various types of data, to/from and with other apparatuses and devices or the like connected via the network NW. For example, the communication I/F 9c3 is configured to transmit the medical information output from the transmission data generating unit 9c2 to the medical information server Ia. For example, the communication I/F 9c3 may be realized by using a network card, a network adaptor, or the like.

Further, as illustrated in FIG. 25, a Device-D 9d includes the camera 21, a transmission data generating unit 9d2, and the communication I/F 23. The Device-D 9d and a Device-F 9f have the same configuration as each other except for being mounted in mutually-different transporters. Thus, the configuration of the Device-D will be explained below, and detailed explanations of the Device-F will be omitted.

The transmission data generating unit 9d2 is configured to assign a data ID and a patient Gr to the data of the images obtained by the camera 21, as identification information. Further, the transmission data generating unit 9d2 is configured to obtain position information from the GPS mounted in the ambulance V1 and to append the position information to the data of the images. Further, the transmission data generating unit 9d2 is configured to generate medical information to be transmitted to the medical information server Ia, by assigning the Device ID to the data of the images and to transmit the medical information to the medical information server Ia via the communication I/F 23.

As illustrated in FIG. 25, the medical information server Ia includes the communication I/F 31, the input I/F 32, the display 33, a storage I4, and a processing circuit I5. The medical information server Ia is an example of the storage device.

The storage I4 is connected to the processing circuit I5 and is configured to store therein various types of data. The storage I4 stores therein a medical information table I41, an application log I42, and a device table I43, for example.

The medical information table I41 stores therein information related to the medical information obtained from the Devices -A to -F. For example, the medical information table I41 stores therein information illustrated in FIG. 24. In the present embodiment, the medical information table I41 is set and updated by an obtaining function I51 and a registering function I52 (explained later), for example.

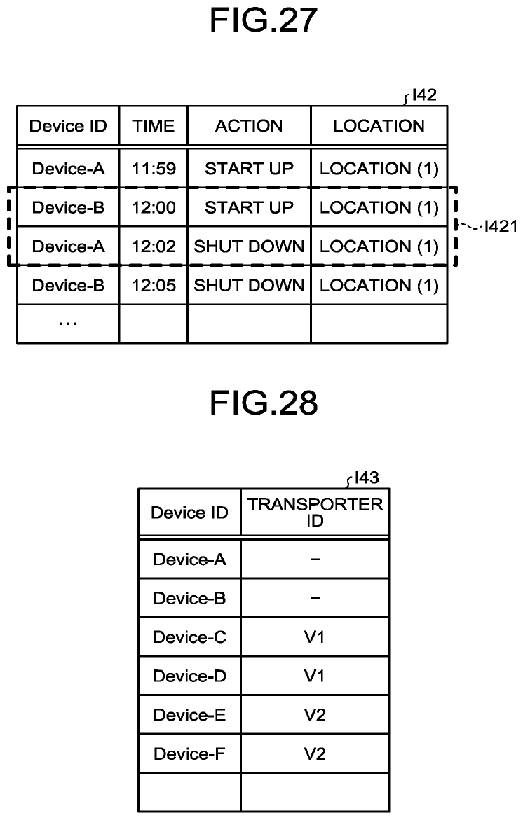

The application log I42 stores therein information related to a start-up status of each of the applications mounted in the Devices -A to -F and used for transmitting the medical information to the medical information server Ia. FIG. 27 is a drawing illustrating an example of the application log according to the fourth embodiment. As illustrated in FIG. 27, the application log I42 stores therein, for example, “Device IDs”, “times”, “actions”, and “locations” so as to be kept in correspondence with one another. The application log I42 is set by the registering function I52, for example. Similarly to the acquisition times and the acquisition locations of the medical information data illustrated in FIG. 24, the times in the application log I42 in FIG. 27 may each include a date, for example, and the locations may be registered in a different format such as an address or longitude and latitude. Further, when it is impossible to obtain position information at the time of occurrence of an action, the item under “location” may be left blank.

For example, the record in the first line in FIG. 27 indicates that the application in the device identified with the Device ID “Device-A” was started up at “11:59” at “location (1)”. Similarly, the record in the second line in FIG. 27 indicates that the application in the device identified with the Device ID “Device-B” was started up at “12:00” at “location (1)”. Similarly, the record in the third line in FIG. 27 indicates that the application in the device identified with the Device ID “Device-A” was shut down at “12:02” at “location (1)”. The record in the fourth line in FIG. 27 indicates that the application in the device identified with the Device ID “Device-B” was shut down at “12:05” at “location (1)”.

The device table I43 stores therein correspondence relationships between the devices and the transporters in which the devices are mounted. FIG. 28 is a drawing illustrating an example of the device table according to the fourth embodiment. As illustrated in FIG. 28, the device table I43 stores therein “Device IDs” so as to be kept in correspondence with “transporter IDs”, for example. The device table I43 is, for example set by an administrator (not illustrated) of the medical information server Ia.

In FIG. 28, the “transporter IDs” are each an identifier uniquely identifying a transporter such as an ambulance or a medical emergency helicopter. In the device table I43 in FIG. 28, each of the devices mounted in a transporter has a transporter ID stored in correspondence with the Device ID, while other devices has no transporter ID registered. For example, in the device table I43 in FIG. 28, no transporter ID is kept in correspondence with the Device IDs “Device-A” and “Device-B”. In other words, the device table I43 in FIG. 28 indicates that the device identified with the Device ID “Device-A” and the device identified with the Device ID “Device-B” are each not mounted in a transporter. Further, the device table I43 in FIG. 28 indicates that the device identified with the Device ID “Device-C” and the device identified with the Device ID “Device-D” are mounted in the ambulance V1. Similarly, the device table 143 in FIG. 28 indicates that the device identified with the Device ID “Device-E” and the device identified with the Device ID “Device-F” are mounted in the medical emergency helicopter V2. As illustrated in FIG. 28, in the present embodiment, two or more Device IDs may be kept in correspondence with one transporter ID, in some situations.

Returning to the description of FIG. 25, the processing circuit I5 is configured to control operations of the entirety of the medical information server Ia, by executing the obtaining function I51 and the registering function I52.

The obtaining function I51 included in the processing circuit I5 is configured to obtain medical information from each of the devices identified as Devices -A to -F. For example, when having received medical information including data of images from any of the devices identified as the Device-A, the Device-B, the Device-D, or the Device-F via the network NW, the obtaining function I51 is configured to store the data of the images into the storage I4 and to also output information such as the data IDs, the Device ID, the data ID, the acquisition time, the acquisition location, and the patient Gr that are kept in association with the medical information, to the registering function I52. Similarly, when having received medical information including data related to a heart rate from either of the devices identified as the Device-C or the Device-E via the network NW, the obtaining function I51 is configured to store the data related to the heart rate into the storage I4 and to also output information such as the data IDs, the Device ID, the data ID, the acquisition time, the acquisition location, and the patient Gr that are kept in association with the medical information, to the registering function I52.

The registering function I52 is configured to register information related to the medical information into the medical information table I41 and to also update the patient Gr registered in the medical information table I41. For example, the registering function I52 is configured to register the data IDs, the device Id, the acquisition time, the acquisition location, and the patient Gr output from the obtaining function I51, into the image table I41.

Further, the registering function I52 is configured to update the patient Gr kept in correspondence with the pieces of medical information, on the basis of the information registered in the medical information table I41. For example, the registering function I52 is configured to extract such pieces of medical information that were acquired by mutually-different devices and registered in the medical information table I41 and to judge whether or not the acquisition times and the acquisition locations of the pieces of medical information are the same as or close to each other. For example, the registering function I52 is configured to judge whether or not the difference in acquisition times between two pieces of medical information is “shorter than 5 minutes” and whether or not the distance between the acquisition locations is “shorter than 50 meters”. When having determined that the acquisition time and the acquisition location of a piece of medical information is the same as or close to the acquisition time and the acquisition location of another piece of medical information, the registering function I52 is configured to update, for example, the patient Gr of the latter piece of medical information with the patient Gr of the former piece of medical information.

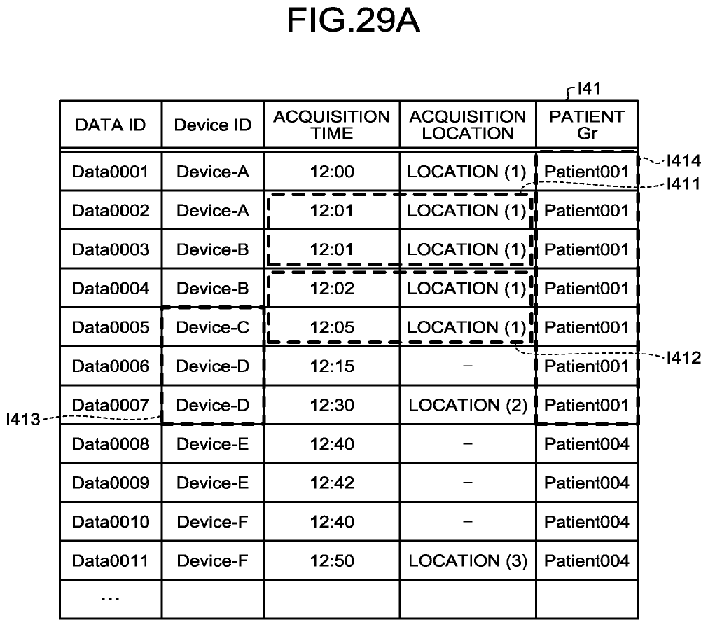

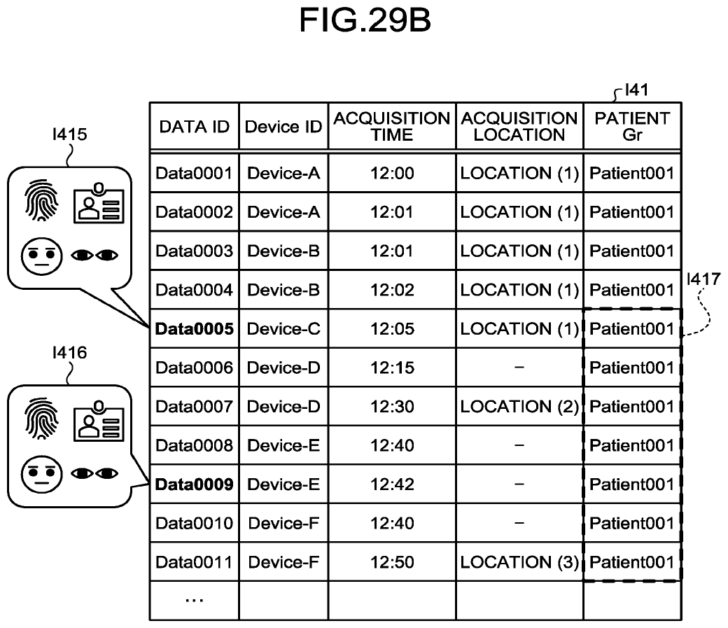

The process performed by the registering function I52 will be explained, with reference to FIGS. 29A and 29B. FIGS. 29A and 29B are drawings illustrating examples of an image table according to the fourth embodiment. In the medical information illustrated in FIG. 29A, for example, the registering function I52 compares the acquisition times and the acquisition locations between the medical information identified with the data ID “Data0002” acquired by the Device-A and the medical information identified with the data ID “Data0003” acquired by the Device-B. In this situation, as indicated with the reference characters I411 in FIG. 29A, the acquisition times “12:01” are the same, and the acquisition locations “location (1)” are the same. Accordingly, it is conjectured that the medical information identified with the data ID “Data0002” and the medical information identified with the data ID “Data0003” are related to the same patient. In that situation, the registering function I52 updates the patient Gr of the data ID “Data0003” with the patient Gr “Patient001” of the data ID “Data0002”.

Subsequently, in the medical information illustrated in FIG. 29A, the registering function I52 compares the acquisition times and the acquisition locations between the medical information identified with the data ID “Data0004” acquired by the Device-B and the medical information identified with the data ID “Data0005” acquired by the Device-C. In that situation, as indicated with the reference characters I412 in FIG. 29A, the acquisition times “12:02” and “12:05” are close to each other, while the acquisition locations “location (1)” are the same. Accordingly, it is conjectured that the medical information identified with the data ID “Data0004” and the medical information identified with the data ID “Data0005” are related to the same patient. In that situation, the registering function I52 updates the patient Gr of the data ID “Data0005” with the updated patient Gr “Patient001” of the data ID “Data0004”.

Further, by referring to the device table I43 illustrated in FIG. 28, the registering function I52 specifies that the device identified with the Device ID “Device-C” and the device identified with the Device ID “Device-D” indicated with the reference characters I413 in FIG. 29A are mounted in the same transporter, namely, the ambulance V1. In that situation, it is conjectured that the medical information acquired by the device identified as the Device-C and the medical information acquired by the device identified as the Device-D are related to the same patient. In this situation, the registering function I52 updates the patient Gr of the medical information identified with the data ID “Data0007” acquired by the device identified as the Device-D, with the updated patient Gr “Patient001” of the data ID “Data0005”.

On the other hand, in the medical information illustrated in FIG. 24, for example, the registering function 152 compares the acquisition time and the acquisition location of the medical information identified with the data ID “Data0007” with the acquisition time and the acquisition location of the medical information identified with the data ID “Data0008”. In that situation, the acquisition times of the two pieces of medical information are “12:30” and “12:40”, which are not close to each other. Also, the acquisition locations of the two pieces of medical information are “location (2)” and “unidentified”, which are conjectured as not being close to each other. In that situation, it is unclear whether it is possible to conjecture that the medical information identified with the data ID “Data0007” and the medical information identified with the data ID “Data0008” are related to the same patient. In this situation, the registering function I52 does not update the patient Gr of the data ID “Data0008”.

As a result of the processes performed by the registering function I52 explained above, the patient Gr of certain pieces of medical information is updated as indicated with the reference characters I414 in FIG. 29A. In other words, as information indicating that it is conjectured that the certain pieces of medical information obtained by Devices -A to -D were obtained from the same patient, the same patient GR was set with those pieces of medical information.

The information used in the judging process by the registering function I52 does not necessarily have to be the acquisition times and the acquisition locations at which the medical information was obtained. For example, it is also acceptable to use the time or the location at which the application installed in each device was started up or shut down. For example, as indicated with the reference characters I421 in FIG. 27, the application in the device identified with the Device ID “Device-B” was started up two minutes before the application in the device identified with the Device ID “Device-A” was shut down, at “location (1)” which is the same as the location at which the application in the device identified as the “Device-A” was started up and shut down. In that situation, it is conjectured that the device identified as the “Device-A” had obtained medical information of the patient before the device identified as the “Device-B” arrived, and the obtainment of the medical information of the patient was thereafter handed over to the device identified as the “Device-B”. In that situation, the registering function I52 may update the patient Gr of the Device ID “Device-B” with the patient Gr “Patient001” of the Device ID “Device-A”.

Further, when the medical information includes information that makes it possible to specify an individual patient, such as biological information of the patient (e.g., a fingerprint, the iris, etc.) or identification information assigned to the patient, the registering function I52 further judges whether or not the information specifying the individual patient matches between two or more pieces of medical information. When having determined that information specifying an individual patient included in a piece of medical information matches information specifying an individual patient included in another piece of medical information, the registering function I52 updates the patient Gr of the latter piece of medical information with the patient Gr of the former piece of medical information. In the following sections, the information specifying an individual patient may simply be referred to as “individual specifying information”. Each of the individual specifying information is an example of patient specifying information.

In the example in FIG. 29B, the medical information “Data0005” acquired by the device identified as the “Device-C” includes individual specifying information I415 such as a fingerprint, the irises, and the ID of the patient. Similarly, the medical information “Data0009” acquired by the device identified as the “Device-E” includes individual specifying information I416. In that situation, the registering function I52 judges whether or not the individual specifying information I415 and the individual specifying information I416 match each other. When having determined that the individual specifying information I415 and the individual specifying information I416 match each other, the registering function I52 updates, as indicated with the reference characters I417 in FIG. 29B, the patient Gr corresponding to the medical information acquired by either the Device E or the Device-F mounted in the same medical emergency helicopter V2 as the Device-E is, with the patient Gr of the medical information acquired by the Device-C. As a result, the registering function I52 is able to set the patient Gr indicating that the certain pieces of medical information acquired from the devices identified as Devices -A to -F were obtained from the same patient.

When judging whether or not pieces of medical information were obtained from the same patient, there is a lower possibility of misidentification of the patients when the judgment is made on the basis of the individual specifying information than when the judgment is made on the basis of the times and the locations. Accordingly, another arrangement is also acceptable in which the registering function I52 does not update the patient Gr when having determined that two pieces of individual specifying information do not match, regardless of the judgment result based on the times and the locations.

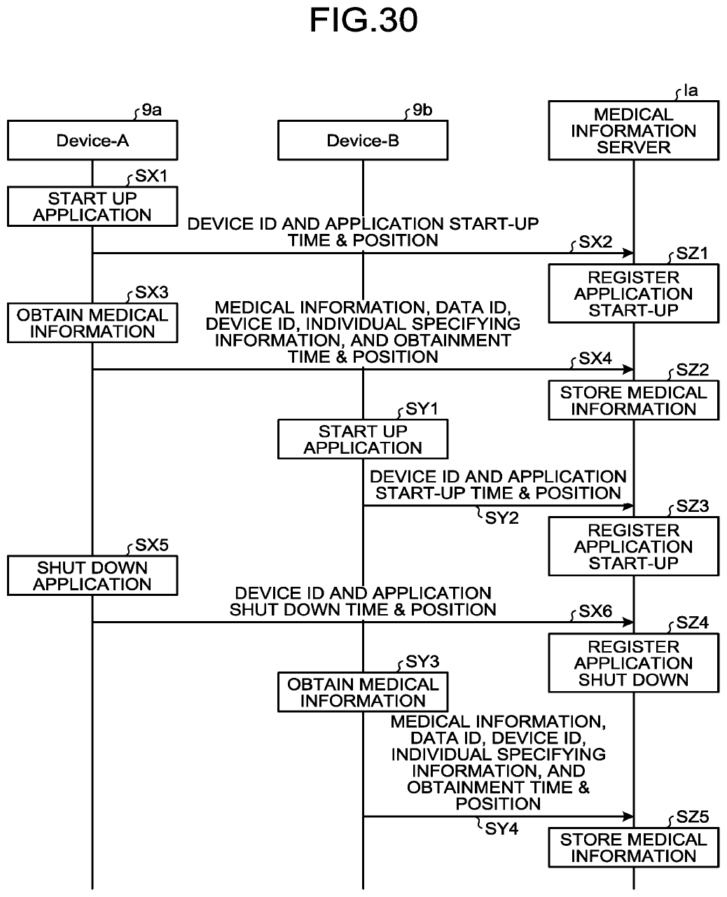

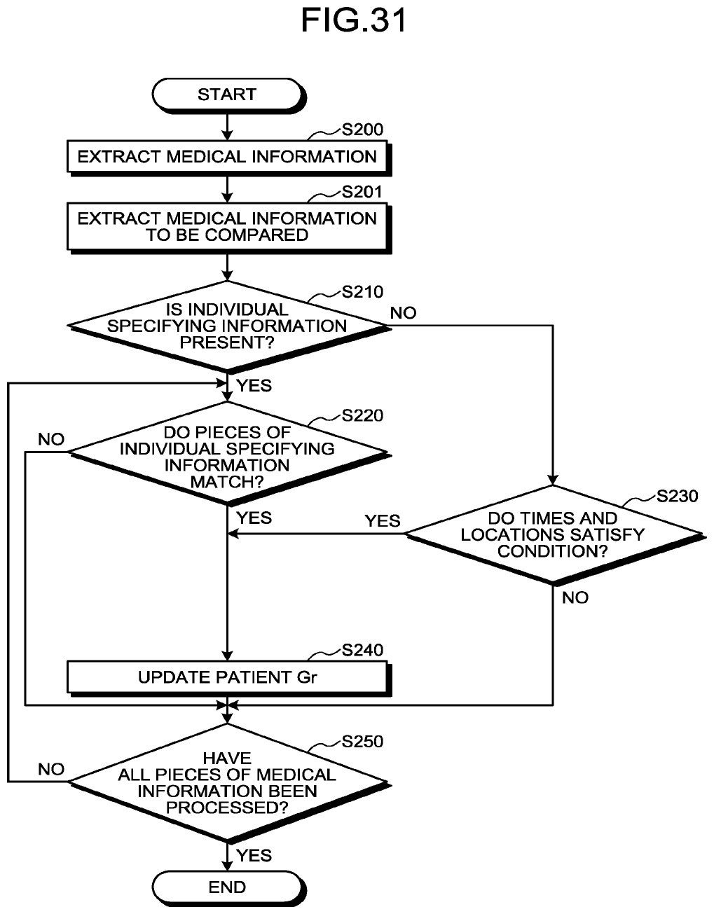

Next, a processing procedure performed by the medical information management system 4 according to the fourth embodiment will be explained, with reference to FIGS. 30 and 31. FIG. 30 is a sequence chart illustrating an example of a medical information obtaining process according to the fourth embodiment. Although FIG. 30 illustrates only two devices, namely the Device-A and the Device-B, another configuration is also acceptable in which the same processes are performed on Devices -C to -F.

For example, as illustrated in FIG. 30, in the medical information management system 4 in the present embodiment, for example, the Device-A 9a (e.g., a smartphone) at first starts up an application used for transmitting medical information to the medical information server Ia, prior to arrival of the Device-B 9b (e.g., a drone) (step SX1) and transmits the Device ID and the time and the position at which the application was started up, to the medical information server Ia (step SX2). The medical information server Ia receives the Device ID and the time and the position at which the application was started up and registers the start-up of the application by the Device-A 9a, into the application log I42 (step SZ1).

Subsequently, when having obtained medical information of the patient (step SX3), the Device-A 9a transmits medical information including the Device ID, data ID, individual specifying information, and the time and the position at which the medical information was obtained, to the medical information server Ia (step SX4). The medical information server Ia registers information related to the received medical information into the medical information table I41 (step SZ2).

After that, when the Device-B 9b starts up an application used for transmitting medical information to the medical information server Ia (step SY1), the Device-B 9b transmits the Device ID and the time and the position at which the application was started up, to the medical information server Ia (step SY2). The medical information server Ia receives the Device ID and the time and the position at which the application was started up, and registers the start-up of the application by the Device-B 9b into the application log I42 (step SZ3).

Further, when the Device-A 9a shuts down the application (step SX5), the Device-A 9a transmits the Device ID and the time and the position at which the application was shut down to the medical information server Ia (step SX6). The medical information server Ia receives the Device ID and the time and the position at which the application was shut down and further registers the shut-down of the application by the Device-A 9a into the application log I42 (step SZ4).

Subsequently, when the Device-B 9b obtains medical information of the patient (step SY3), the Device-B 9b transmits medical information including the Device ID, data ID, individual specifying information, and the time and the position at which the medical information was obtained, to the medical information server Ia (step SY4). The medical information server Ia registers information related to the received medical information into the medical information table I41 (step SZ5).

As a result of the processes described above, the information illustrated in FIG. 24 has been registered in the medical information table I41, and also, the information illustrated in FIG. 27 has been registered in the application log I42.

Next, a process performed by the medical information server Ia to identifying a plurality of pieces of medical information concerning to a same patient by updating the information registered in the medical information table I41 will be explained. FIG. 31 is a flowchart illustrating an example of the medical information updating process according to the fourth embodiment. In this situation, steps S200 through S250 illustrated in FIG. 31 are realized, for example, as a result of the processing circuit I5 included in the medical information server Ia reading and executing the program corresponding to the registering function I52 from the storage I4. Further, the medical information updating process illustrated in FIG. 31 is performed with arbitrary timing such as, for example, at the time when a new piece of medical information is received or at the time when a medical information obtainment request is received from the management apparatus Ga. the medical information server Ia updates the medical information table I41 at each time when a new piece of medical information is transmitted from any of the extra-order devices (Devices -A to -F), all pieces of the medical information stored in the medical information table 141 had been already identified with a same patient when the patient arrived at the Hospital HPa. Consequently, the management apparatus Ga is able to associate all pieces of the medical information with patient identification information corresponding to an order as a single package.

At first, the processing circuit I5 extracts medical information subject to the process, from the medical information table I41 registering therein the information illustrated in FIG. 24 (step S200). Subsequently, the processing circuit I5 extracts medical information to be compared, from the medical information table I41 (step S201).

Subsequently, the processing circuit I5 judges whether or not the pieces of medical information each include individual specifying information (step S210). When having determined that both of the pieces of medical information includes individual specifying information (step S210: Yes), the processing circuit I5 judges whether or not the pieces of individual specifying information included in the pieces of medical information match each other (step S220).

When having determined that the pieces of individual specifying information match each other (step S220: Yes), the processing circuit I5 updates the patient Gr corresponding to the medical information (step S240) and proceeds to step S250. On the contrary, when having determined that the pieces of individual specifying information do not match (step S220: No), the processing circuit I5 proceeds to step S250.

Returning to the description of step S210, when at least one of the pieces of medical information does not include any individual specifying information (step S210: No), the processing circuit I5 judges whether the times and the locations included in the pieces of medical information satisfy the condition of being the same as or close to one another (step S230). When having determined that the condition is satisfied (step S230: Yes), the processing circuit I5 updates the patient Gr corresponding to the medical information (step S240) and proceeds to step S250. On the contrary, when having determined that the condition is not satisfied (step S230: No), the processing circuit I5 proceeds to step S250.

After that, the processing circuit I5 judges whether or not the process has been completed with respect to all the pieces of medical information (step S250). When having determined that the process has not been completed (step S250: No), the processing circuit I5 returns to step S200 and repeats the process. On the contrary, when having determined that the process has been completed (step S250: Yes), the processing circuit I5 ends the process.

As explained above, according to the fourth embodiment, the processing circuit I5 included in the storage device (the medical information server Ia) is configured to judge whether or not the times (the acquisition times) and the locations (the acquisition locations) at which certain pieces of medical information were acquired by mutually-different extra-order device (Devices -A to -F) are the same as or close to one another. When it is determined that the times and the locations are the same as or close to one another, the processing circuit I5 is configured to update the issued identification information (the patient Gr) corresponding to at least one of the pieces of medical information. Further, the processing circuit G5 included in the medical information management apparatus is configured to bring the medical information of which the issued identification information has been updated, into association with the patient identification information (the emergency ID). The medical information management system 4 according to the fourth embodiment is able to easily bring the medical information obtained by the extra-order devices into association, without depending on the transport IDs or the Device IDs. Consequently, the medical information management system 4 according to the fourth embodiment is able to easily identify a plurality of pieces of medical information concerning to a same patient acquired by a same or a different extra-order device.

Further, according to the fourth embodiment, the processing circuit I5 included in the storage device is configured to judge whether nor not the pieces of information specifying the patients (the individual specifying information) and being included in the pieces of medical information obtained by the mutually-different extra-order devices (the Devices -A to -F) match one another. When it is determined that certain pieces of information specifying the patients match one another, the processing circuit I5 may update the issued identification information (the patient Gr) corresponding to at least one of the pieces of medical information. The processing circuit G5 of the medical information management apparatus is configured to bring the medical information of which the issued identification information has been updated, into association with the patient identification information. With this arrangement, the medical information management system 4 according to the fourth embodiment is able to bring the medical information obtained by the extra-order devices into association accurately.

Further, the extra-order devices may be extra-order devices (the Devices -C to -F) that are kept in association with the transporters (the ambulance V1 and the medical emergency helicopter V2) that transport patients to the hospital.

Further, when a single device has obtained medical information of a plurality of patients in case of a massive disaster or the like, another configuration is acceptable in which the patient Gr is not updated on the basis of the times and the locations because there is a high possibility that the patients may be misidentified.

Further, an extra-order device is not limited to an extra-hospital device such as a device associated with a transporter, a smartphone, and a drone. The extra-order device may be another devices mounted in the hospital HP. Moreover, medical information acquired by the modalities mounted in the hospital HP without following a medical order issued by the hospital HP is treated as same as the medical information acquired by extra-order devices.

Further, the example is explained above in which the application used for transmitting the medical information is installed in each of the external devices, namely, the Device-A and the Device-B; however, possible embodiments are not limited to this example. For instance, in each of the Devices -C to -F, which are onboard devices, it is also acceptable to install the same application as those installed in the Device-A and the Device-B.

Further, the example is explained above in which the registering function I52 updates the patient Gr, by handing over the patient Gr corresponding to the medical information obtained earlier to the patient Gr corresponding to the medical information obtained later; however, possible embodiments are not limited to this example. It is also acceptable to configure the registering function I52 to issue a new patient Gr.

Further, the data stored in the PACSs 50 do not necessarily have to be in the DICOM format. For example, the data may be in an HL7 format used in an electronic medical record system, for example. Further, as long as the emergency IDs are each able to uniquely identify a patient at the hospital HPa, the emergency IDs do not necessarily have to be newly issued by the RIS 40a, for example. Each of the emergency IDs may be patient identification information or the like that has already been registered.

The term “processor” used in the description of the above embodiments denotes, for example, a Central Processing Unit (CPU), a Graphics Processing Unit (GPU), or a circuit such as an Application Specific Integrated Circuit (ASIC) or a programmable logic device (e.g., a Simple Programmable Logic Device [SPLD], a Complex Programmable Logic Device [CPLD], or a Field Programmable Gate Array [FPGA]). In this situation, instead of saving the programs in a memory, it is also acceptable to directly incorporate the programs in the circuits of the processors. In that situation, the processors realize the functions by reading and executing the programs incorporated in the circuits thereof. The processors in the present embodiment do not each necessarily have to be structured as a single circuit. It is also acceptable to structure one processor by combining together a plurality of independent circuits so as to realize the functions thereof.

In this situation, the programs executed by the one or more processors are provided as being incorporated, in advance, in a Read-Only Memory, a storage unit, or the like. Alternatively, the programs may be provided as being recorded in a computer-readable storage medium such as a Compact-Disk Read-Only Memory (CD-ROM), a Flexible Disk (FD), a Compact Disk Recordable (CD-R), or a Digital Versatile Disk (DVD), in a file in a format that is installable or executable in those devices. Further, the programs may be provided or distributed by being stored in a computer connected to a network such as the Internet and being downloaded via the network. For example, the programs are each structured with a module including functional units. In actual hardware, as a result of a CPU reading and executing the programs from a storage medium such as a ROM, the modules are loaded into a main storage device and generated in the main storage device.

Further, the configurations of the medical information management systems in the embodiments are not limited to those described above. For example, another computer such as the RIS 40, the PACS 50, or the like may have a part or all of the functions of the processing circuit 15 installed therein or may have a part or all of the content of the storage 14 stored therein. Further, the functions of the processing circuit 15 included in the management apparatus 10 may be installed in a cloud, and the content of the storage 14 may be stored in a cloud.

According to at least one aspect of the embodiments described above, it is possible to easily bring the data related to the patient acquired without following an order, into association at the hospital being the transport destination.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information management system comprising:
a storage device storing therein pieces of medical information of patients acquired by an extra-order device without following a medical order issued by a hospital; and
a medical information management apparatus configured to obtain medical information of a patient who is to use the hospital, from among the pieces of medical information stored in the storage device, wherein
the medical information management apparatus includes a processing circuit configured to:
obtain the medical information of the patient who is to use the hospital, from among the pieces of medical information stored in the storage device; and
obtain patient identification information uniquely identifying, within the hospital, the patient who is to use the hospital, and bring the obtained medical information of the patient into association with the patient identification information.

2. The medical information management system according to claim 1, wherein
the storage device stores therein identification information of the extra-order device, an arrival status at the hospital, and medical information acquired by the extra-order device, so as to be kept in correspondence with one another, and
the processing circuit included in the medical information management apparatus obtains the identification information of the extra-order device and the arrival status at the hospital, and obtains the medical information associated with the identification information of the extra-order device and the arrival status at the hospital, as medical information of the patient who is to use the hospital.

3. The medical information management system according to claim 1, wherein
the storage device stores therein issued identification information issued by the extra-order device and uniquely identifying a patient who used the extra-order device and medical information acquired by the extra-order device, so as to be kept in correspondence with each other, and
the processing circuit included in the medical information management apparatus obtains the issued identification information and further obtains the medical information kept in correspondence with the issued identification information as the medical information of the patient who is to use the hospital.

4. The medical information management system according to claim 3, wherein
the extra-order device receives the issued identification information issued by another extra-order device and handed over thereto,
the storage device stores therein issued identification information uniquely identifying a same patient among two or more extra-order devices and medical information acquired by the extra-order devices, so as to be kept in correspondence with each other, and
the processing circuit included in the medical information management apparatus obtains the issued identification information and further obtains the medical information kept in correspondence with the issued identification information as the medical information of the patient who is to use the hospital.

5. The medical information management system according to claim 1, wherein
the patient identification information is kept in correspondence with a medical order issued by the hospital, and
the processing circuit included in the medical information management apparatus converts the obtained medical information of the patient into a data format same as a data format of medical information that was obtained by following the medical order and is kept in correspondence with the patient identification information and further brings the medical information resulting from the conversion into association with the patient identification information.

6. The medical information management system according to claim 1, wherein
the storage device includes a processing circuit configured to:
identify a plurality of pieces of medical information of a same patient acquired by a same or a different extra-order device, and
the processing circuit included in the medical information management apparatus acquires the identified pieces of medical information of the same patient as medical information of the patient who is to use the hospital, and brings the acquired medical information into association with the patient identification information.

7. The medical information management system according to claim 1, wherein
the storage device includes a processing circuit configured to:
judge whether times and locations at which pieces of medical information were obtained by mutually-different extra-order devices are same as or close to each other; and
update issued identification information corresponding to at least one of the pieces of medical information, when it is determined that the times and the locations at which the pieces of medical information were obtained are the same as or close to each other, and
the processing circuit included in the medical information management apparatus brings the medical information of which the issued identification information has been updated, into association with the patient identification information.

8. The medical information management system according to claim 1, wherein
the storage device includes a processing circuit configured to:
judge whether or not pieces of patient specifying information match each other, the pieces of specifying information each being included in a different one of pieces of medical information obtained by mutually-different extra-order devices; and
update issued identification information corresponding to at least one of the pieces of medical information, when it is determined that the pieces of patient specifying information match each other, and
the processing circuit included in the medical information management apparatus brings the medical information of which the issued identification information has been updated, into association with the patient identification information.

9. The medical information management system according to claim 1, wherein the extra-order device is an extra-order device kept in association with a transporter configured to transport the patient to the hospital.

10. A medical information management apparatus comprising a processing circuit configured to:
obtain medical information of a patient who is to use a hospital, from among pieces of medical information of patients acquired without following a medical order issued by the hospital and stored in a storage device; and
obtain patient identification information uniquely identifying, within the hospital, the patient who is to use the hospital and to further bring the medical information of the patient obtained by the medical information obtaining unit, into association with the patient identification information.

11. A medical information management method implemented by a processing circuit of a medical information management apparatus, the medical information management method comprising:
obtaining medical information of a patient who is to use a hospital, from among pieces of medical of patients acquired without following a medical order issued by the hospital and stored in a storage device; and
obtaining patient identification information uniquely identifying, within the hospital, the patient who is to use the hospital and further bringing the medical information of the patient obtained by the medical information obtaining unit, into association with the patient identification information.
